(19) United States
(12) Patent Application Publication
(10) Pub. No.: US 2014/0039546 A1
(43) Pub. Date: Feb. 6, 2014

(54) ACCESS CLOSURE CONFIGURATION

(71) Applicant: Arstasis Inc., Redwood City, CA (US)

(72) Inventors: D. Bruce Modesitt, San Carlos, CA (US); Joseph F. Paraschac, Campbell, CA (US); Brian A. Ellingwood, Sunnyvale, CA (US)

(73) Assignee: Arstasis Inc., Redwood City, CA (US)

(21) Appl. No.: 13/955,539

(22) Filed: Jul. 31, 2013

Related U.S. Application Data
(60) Provisional application No. 61/678,324, filed on Aug. 1, 2012.

(51) Int. Cl. A61B 17/34

(52) U.S. Cl. CPC A61B 17/3417
USPC 606/213

(57) ABSTRACT

A method may comprise creating a first passage across a wall with a sharpened member at a first angle relative to a lumen; advancing an anchor assembly through the first passage such that a distal portion of the anchor assembly is placed within the blood vessel; applying a force to the anchor assembly to position an adjacent portion of the blood vessel wall into a desired contact configuration relative to the anchor assembly; and while maintaining the desired contact configuration, advancing a needle through a portion of the anchor assembly and through a portion of the wall of the blood vessel to form an expandable tract between overlapping tissue portions of the vessel wall, wherein a portion of the anchor assembly comprises a saddle-shaped needle receiving structure configured to receive and support the needle after it has been advanced across the portion of the wall to create the expandable tract.

### RELATED APPLICATION DATA

The present application claims the benefit under 35 U.S.C. §119 to U.S. Provisional Application Ser. No. 61/678,324, filed Aug. 1, 2012. The foregoing application is hereby incorporated by reference into the present application in its entirety.

### FIELD OF THE INVENTION

The present invention relates to the field of accessing a biological lumen and closing the access pathway or tract thereby created.

### BACKGROUND

A number of diagnostic and interventional vascular procedures are now performed translumenally, where a catheter is introduced to the vascular system at a convenient access location, such as the femoral, brachial, radial, or subclavian arteries, and guided through the vascular system to a target location to perform therapy or diagnosis. When vascular access is no longer required, the catheter and other vascular access devices must be removed from the vascular entrance and bleeding at the puncture site must be stopped. One common approach for providing hemostasis at this site is to apply external force near and upstream from the puncture site, typically by what is known as “manual compression” technique. This hemostasis technique is time-consuming, frequently requiring one-half hour or more of compression before hemostasis. This procedure is uncomfortable for the patient and frequently requires administering analgesics. Excessive pressure can also present the risk of total occlusion of the blood vessel, resulting in ischemia and/or thrombosis. After hemostasis is achieved by manual compression, the patient typically is required to remain recumbent for six to eighteen hours under observation to assure continued hemostasis. During this time bleeding from the vascular access wound can restart potentially resulting in major complications. These complications may require blood transfusion and/or surgical intervention.

Bioabsorbable fasteners have also been used to stop bleeding. Generally, these approaches rely on the placement of a thrombogenic and bioabsorbable material, such as collagen, at the superficial arterial wall over the puncture site. This method generally presents difficulty locating the interface of the overlying-tissue and the adventitial surface of the blood vessel. Implanting the fastener too far from the desired location can result in failure to provide hemostasis. If, however, the fastener intrudes into the vascular lumen, thrombus can form on the fastener. Thrombus can embolize downstream and/or block normal blood flow at the thrombus site. Implanted fasteners can also cause infection and auto-immune reactions/rejections of the implant.

Suturing methods also are used to provide hemostasis after vascular access. The suture-applying device typically is introduced through the tissue tract with a distal end of the device located at the vascular puncture. Needles in the device draw suture through the blood vessel wall on opposite sides of the punctures, and the suture is secured directly over the adventitial surface of the blood vessel wall to close the vascular access wound. To be successful, suturing methods typically need to be performed with a precise control; the associated needles need to be properly directed through the blood vessel wall so that the suture is well anchored in tissue to provide for tight closure. Suturing methods typically also require additional steps for the surgeon, interventionalist, or physician.

Due to the deficiencies of the above methods and devices, a need exists for a more reliable vascular closure configuration and technique. There also exists a need for a vascular closure device and method that is self-sealing and secure. There also exists a need for a vascular closure device and method requiring no or few extra steps to close the vascular site. Configurations are presented herein to address these challenges.

### SUMMARY

One embodiment is directed to a method for forming an expandable tract across a wall of a blood vessel, comprising: creating a first passage across the wall with a sharpened member at a first angle relative to a lumen longitudinal axis defined by a lumen of the blood vessel in the region adjacent the first passage; advancing an anchor assembly through the first passage such that a distal portion of the anchor assembly is placed within the lumen of the blood vessel; applying a force to the anchor assembly to position an adjacent portion of the blood vessel wall into a desired contact configuration relative to the anchor assembly; and while maintaining the desired contact configuration, advancing a needle through a portion of the anchor assembly and through a portion of the wall of the blood vessel to form an expandable tract between overlapping tissue portions of the vessel wall, wherein a portion of the anchor assembly comprises a saddle-shaped needle receiving structure configured to receive and support the needle after it has been advanced across the portion of the wall to create the expandable tract. The sharpened member may comprise a scalpel or a needle. The first passage may be substantially straight in geometry. The anchor assembly may comprise a flexible distal portion removably coupled to a substantially rigid proximal portion, and the flexible distal portion may be advanced through the first passage, after which it may be coupled to the substantially rigid proximal portion and a force is applied to the anchor assembly to position the adjacent portion of the blood vessel wall into the desired contact configuration relative to the anchor assembly. The desired contact configuration may be characterized in that at least a portion of the anchor assembly is oriented substantially parallel to the lumen longitudinal axis. The desired contact configuration may be characterized in that at least a portion of the anchor assembly is oriented substantially parallel to an axis parallel to a portion of the vessel wall immediately adjacent the expandable tract. The method further may comprise controllably extending a load assisting structure from the anchor assembly before applying the force to the anchor assembly. Controllably extending may comprise rotating the load assisting structure about a pivot point relative to the anchor assembly. Controllably extending may comprise extending the load assisting member outward from an outer surface of the anchor assembly along a substantially straight axial pathway relative to the anchor assembly. Controllably extending may comprise extending the load assisting member outward from an outer surface of the anchor assembly along an arcuate pathway relative to the anchor assembly. Controllably extending may comprise manually applying a load to a proximal portion of the anchor assembly, at least a portion of such load being transferred to one or more members coupled to the load assisting structure. The expandable tract may be substantially straight in geometry. The expandable tract may comprise two longitudinal portions that are angled relative to each other. A distal portion leading to the lumen of the blood vessel may be angled more steeply relative to the lumen longitudinal axis than is a more proximal portion. The method further may comprise advancing the needle until a distal portion of the needle comes into contact with at least a portion of the saddle-shaped needle receiving structure. The method further may comprise advancing a guidewire through the expandable tract. The needle may be hollow, and the guidewire may be advanced through a needle lumen defined through the needle. The method further may comprise withdrawing the anchor assembly, leaving behind only the guidewire through the expandable tract. The method further may comprise advancing a dilating instrument across the expandable tract. The method further may comprise advancing one or more elongate instruments through the dilating instrument to conduct a portion of a diagnostic or interventional procedure. The method further may comprise withdrawing the one or more elongate instruments and dilating instrument and leaving a guidewire in place across the expandable tract. The method further may comprise withdrawing the guidewire to allow blood pressure acting on the vessel wall to cause the overlapping tissue portions to collapse against each other and self-seal the expandable tract. The method further may comprise withdrawing the one or more elongate instruments and the guidewire and leaving the dilating instrument in place across the expandable tract.

The method further may comprise withdrawing the dilating instrument to allow blood pressure acting on the vessel wall to cause the overlapping tissue portions to collapse against each other and self-seal the expandable tract. The method further may comprise withdrawing the one or more elongate instruments, the dilating instrument, and the guidewire to allow blood pressure acting on the vessel wall to cause the overlapping tissue portions to collapse against each other and self-seal the expandable tract.

### BRIEF DESCRIPTION OF THE DRAWINGS

FIGS. 1A-1U illustrate schematic representations of one embodiment of a lumen access and closure configuration in accordance with the present invention, at various stages of deployment.

FIGS. 2A-2T illustrate three-dimensional assemblies and subassemblies in accordance with various embodiments of lumen access and closure configurations of the present invention, in various stages of deconstruction.

FIG. 3 illustrates one embodiment of a lumen access and closure technique in accordance with the present invention.

FIG. 4 illustrates one embodiment of a lumen access and closure technique in accordance with the present invention.

FIGS. 5A and 5B illustrate embodiments of single-segmented access tracts created in accordance with the present invention.

FIGS. 6A and 6B illustrate embodiments of single-segmented access tracts created in accordance with the present invention.

FIGS. 7A and 7B illustrate embodiments of single-segmented access tracts created in accordance with the present invention.

FIGS. 8A-8C illustrate three-dimensional assemblies and subassemblies in accordance with various embodiments of lumen access and closure configurations of the present invention, in various stages of deconstruction.

### DETAILED DESCRIPTION

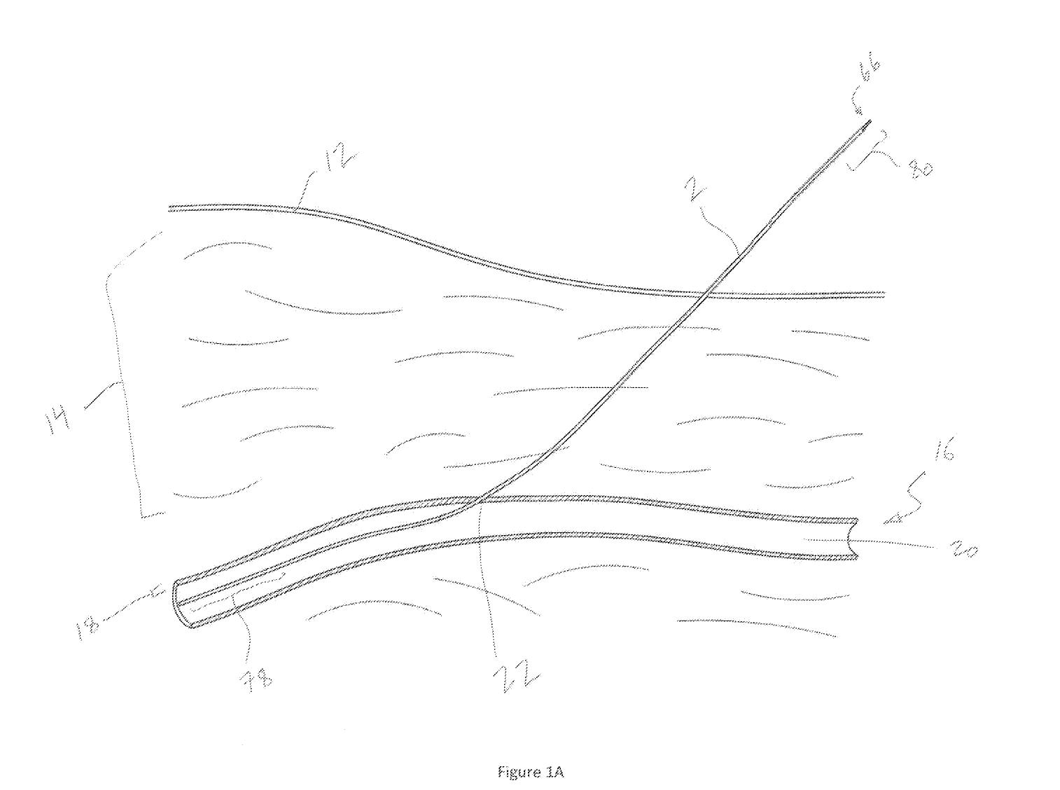

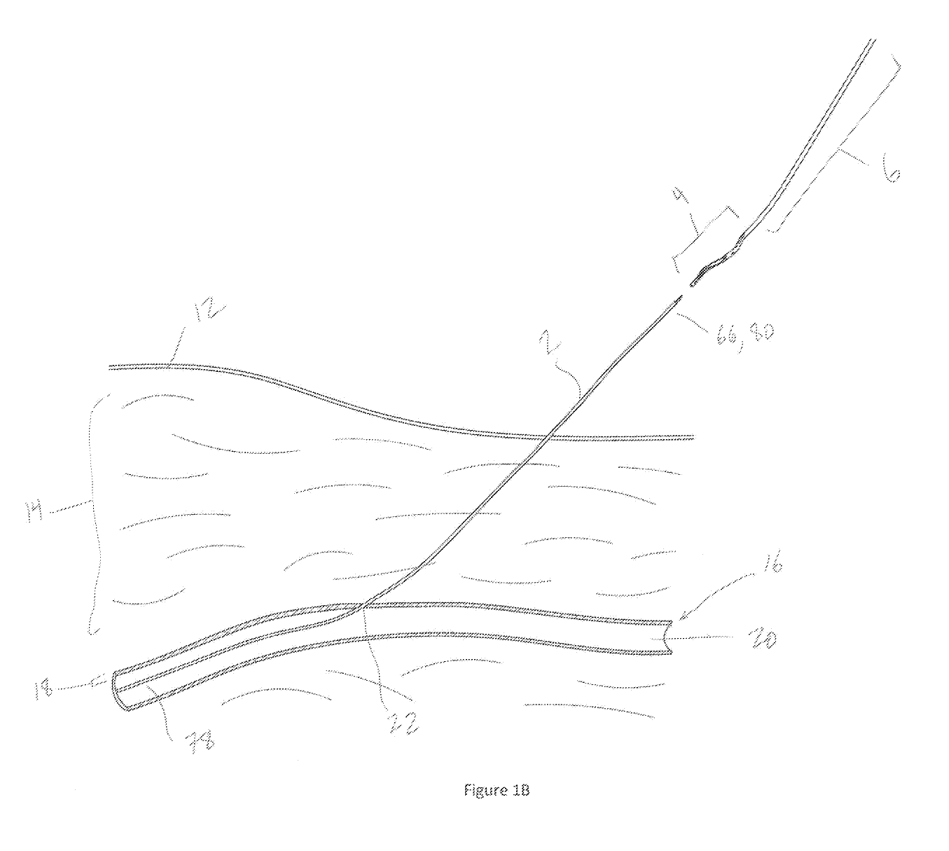

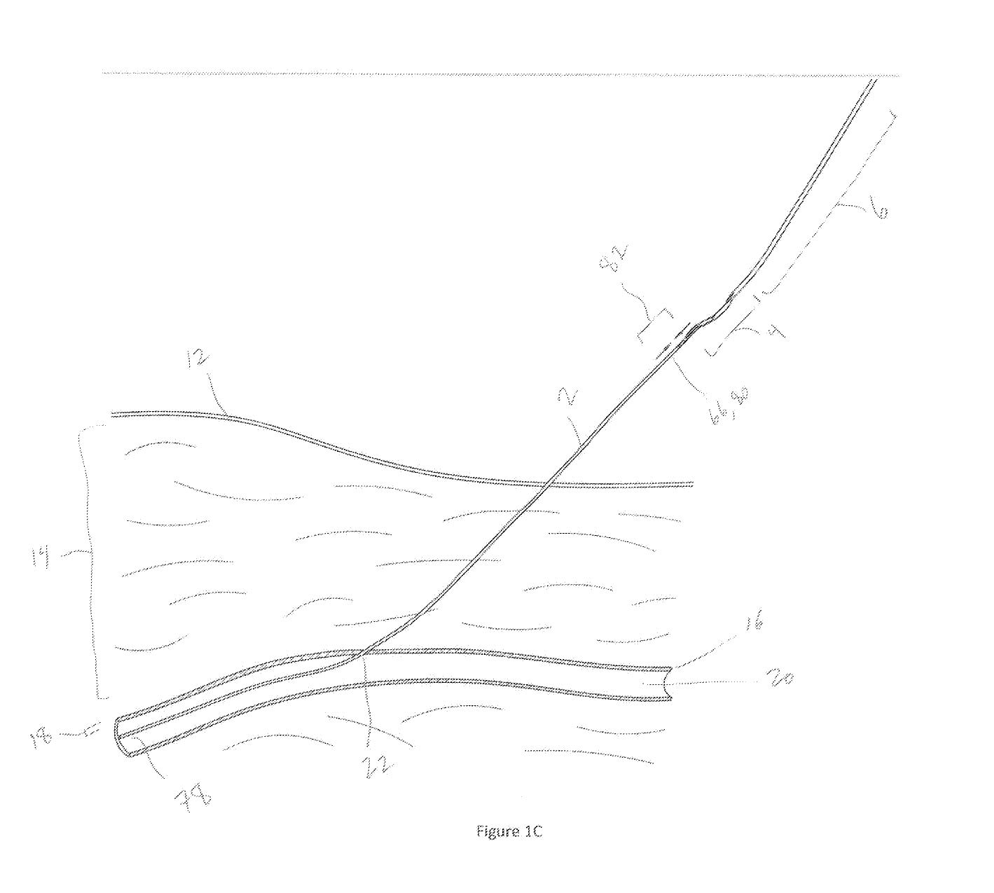

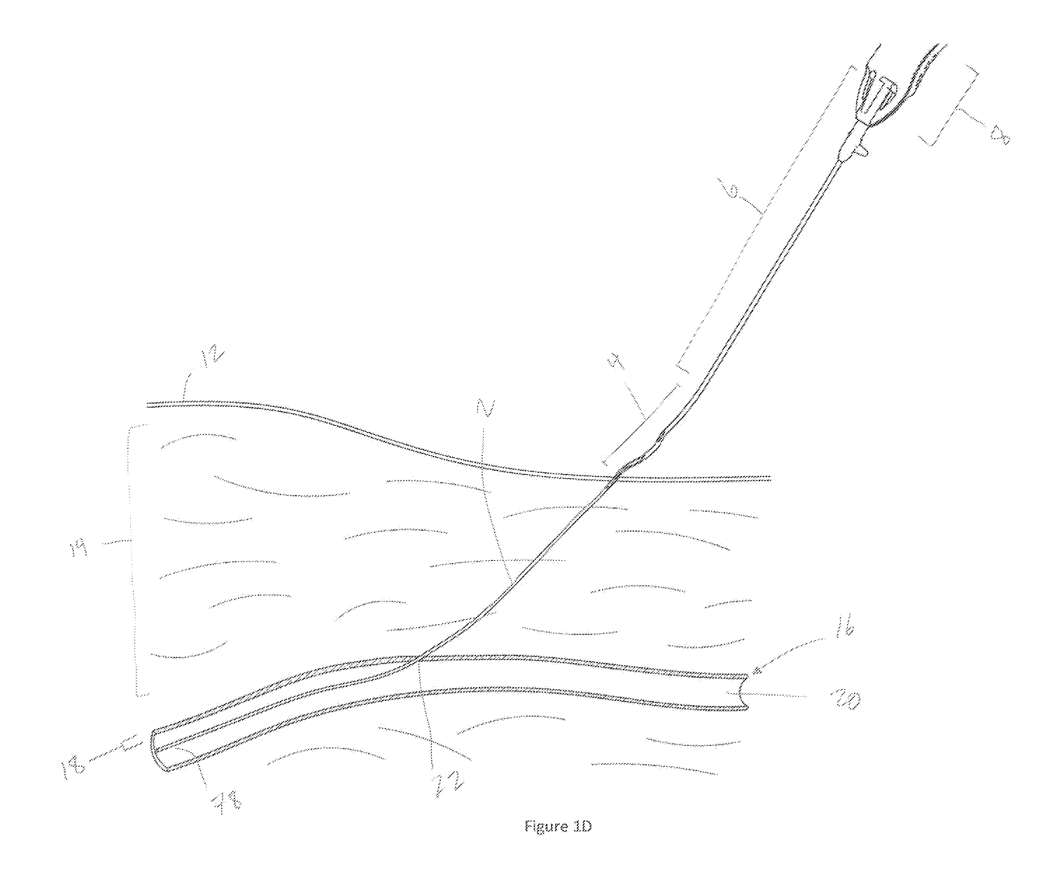

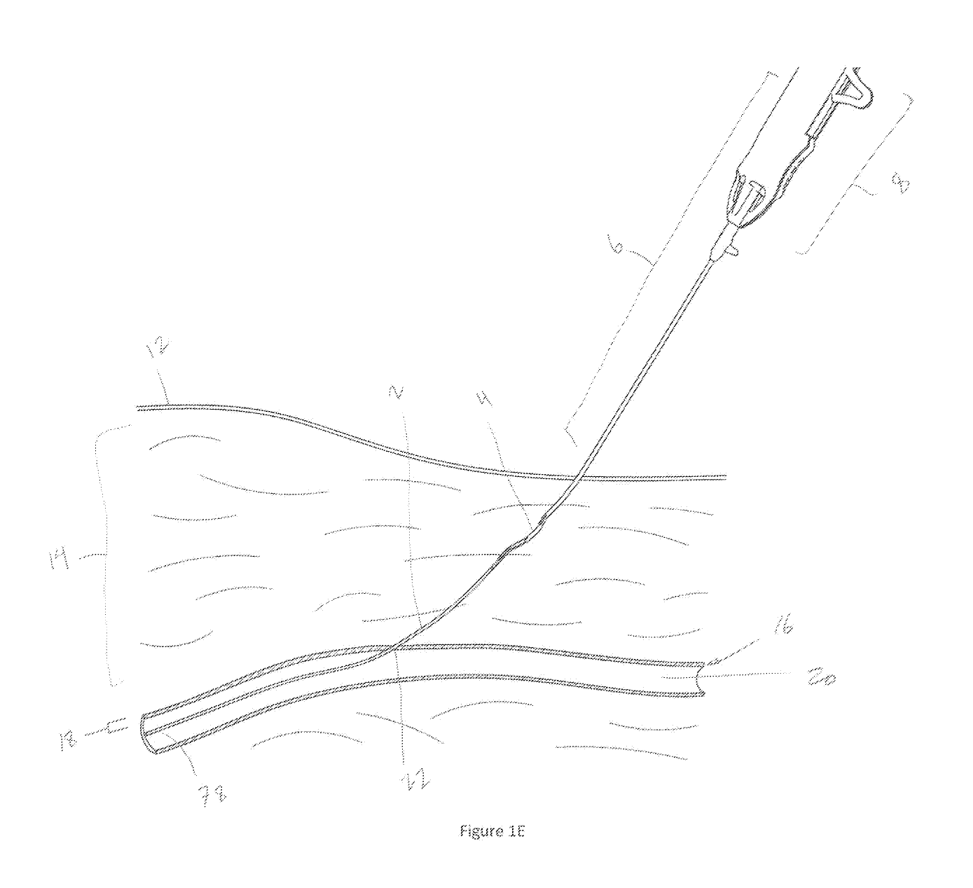

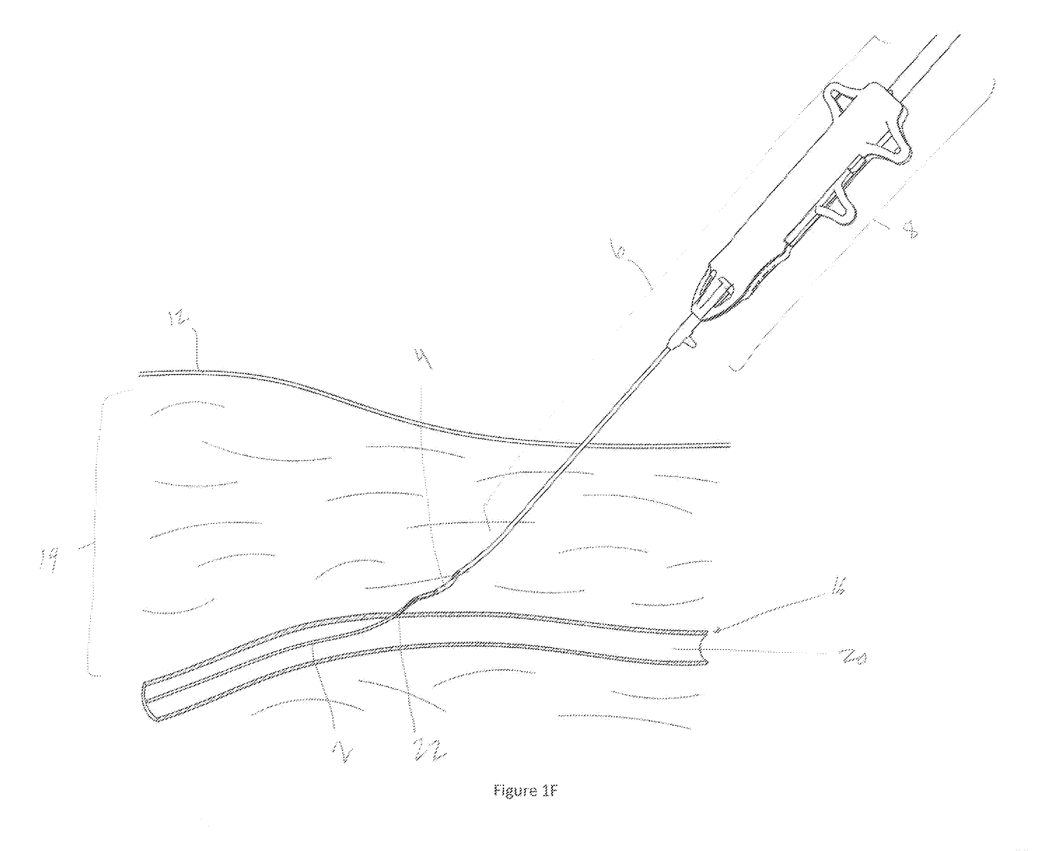

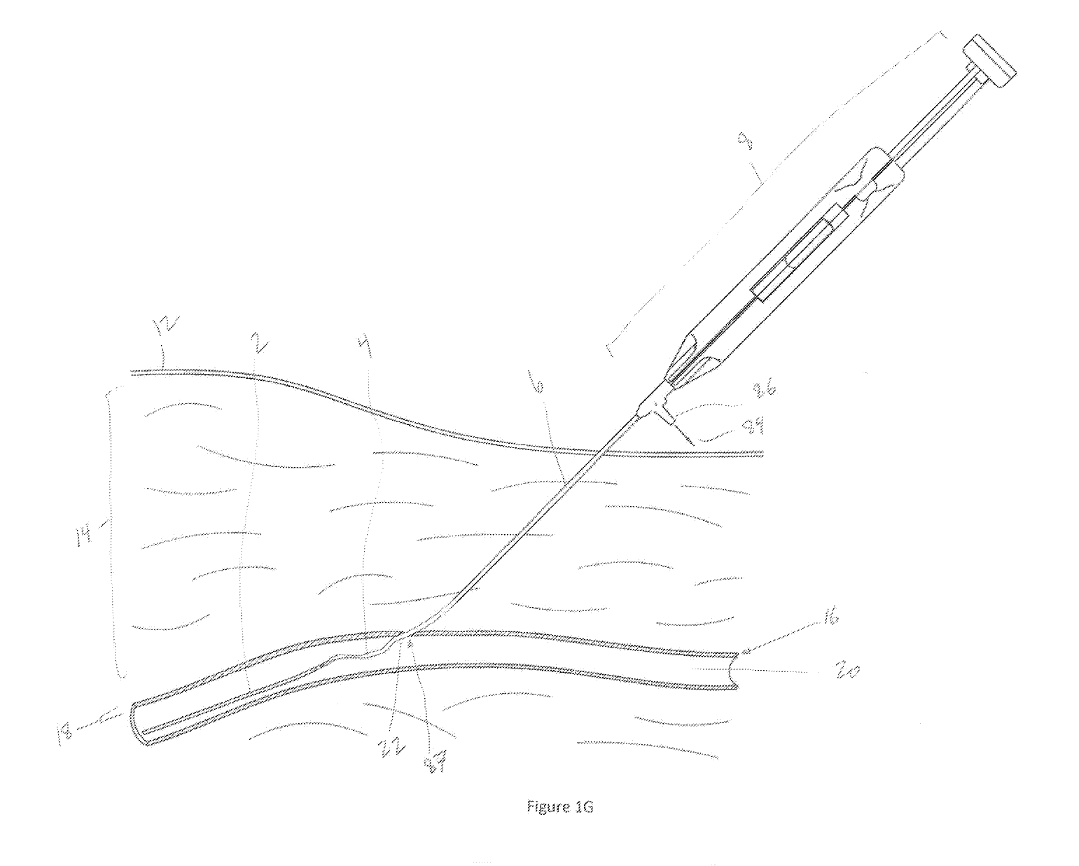

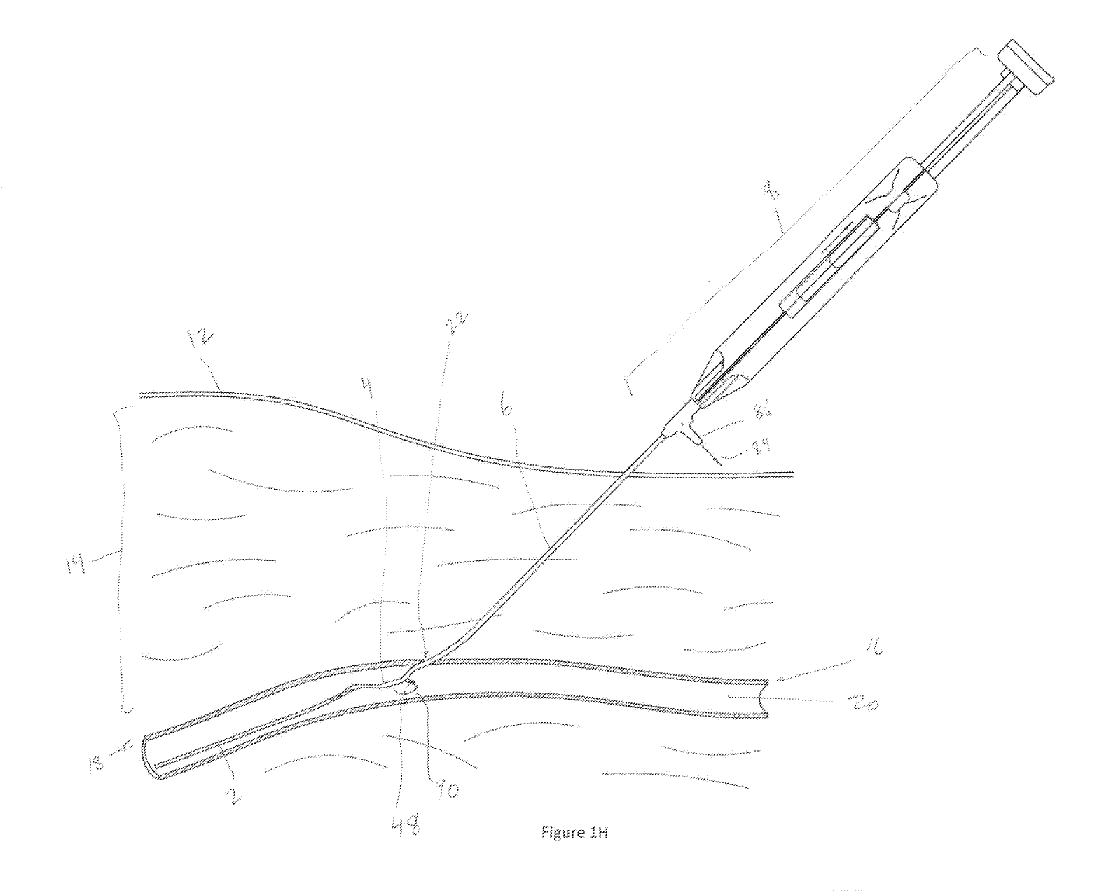

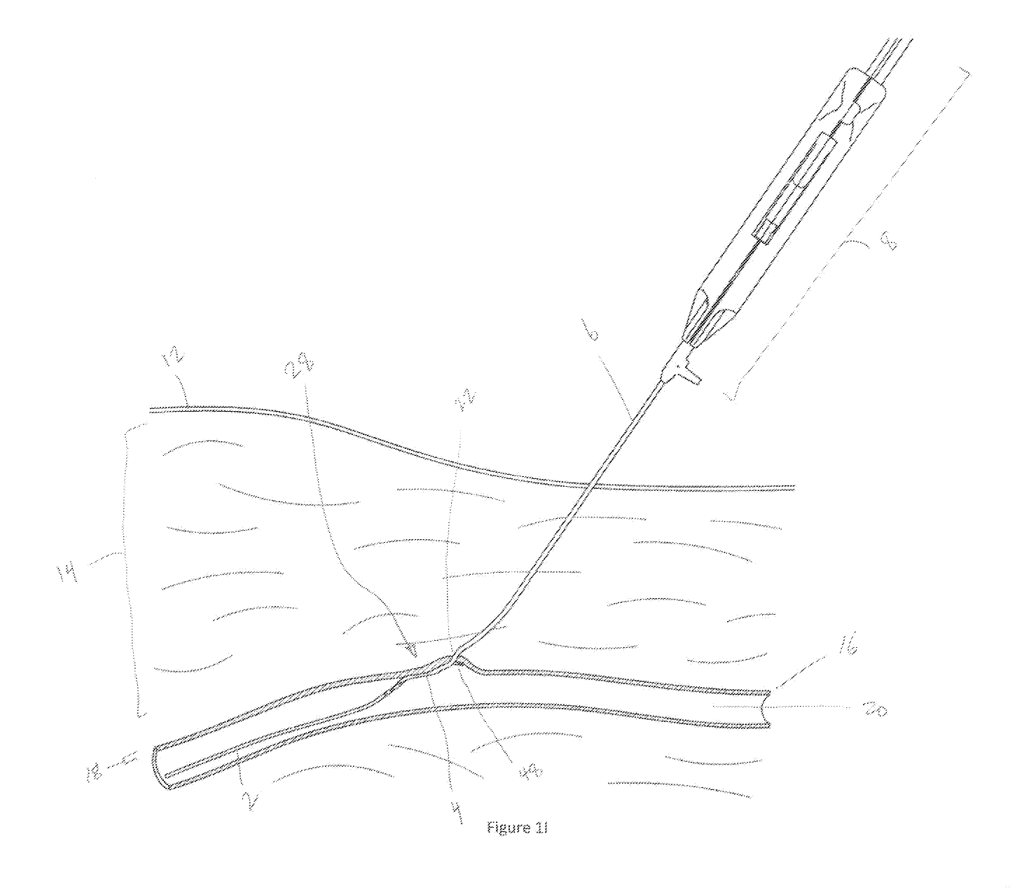

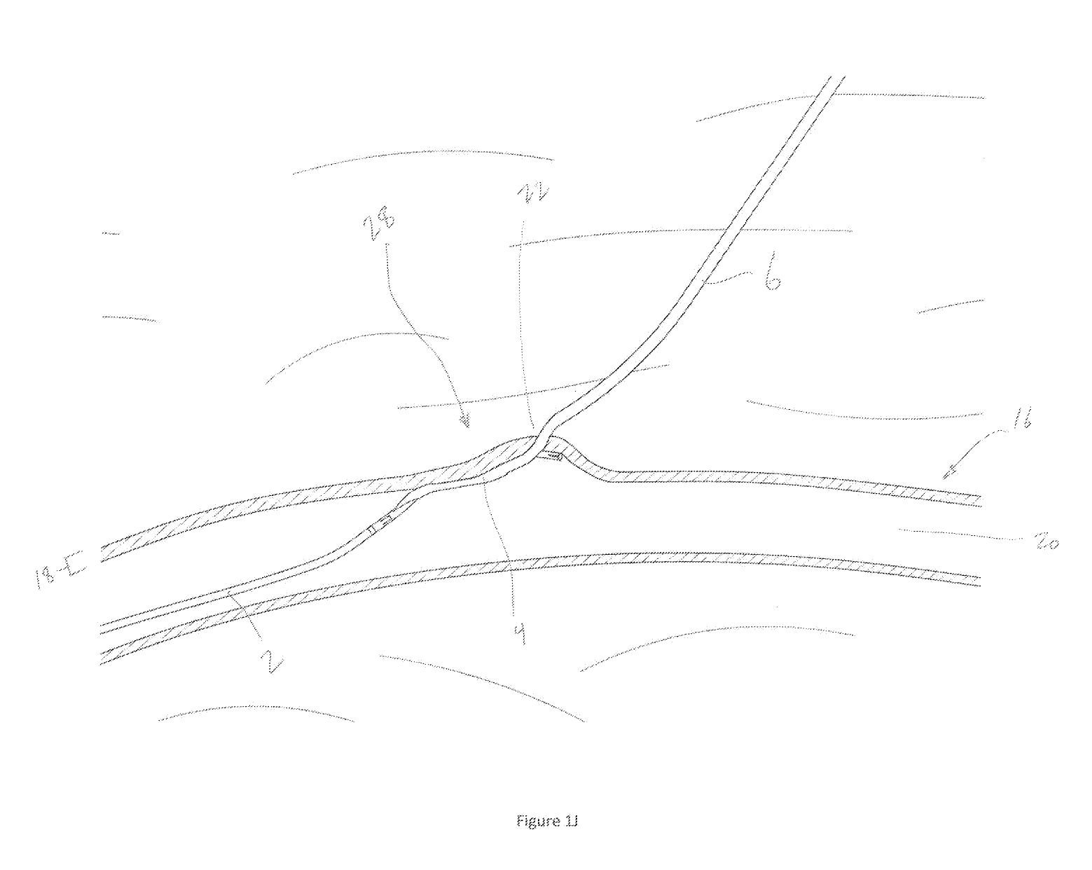

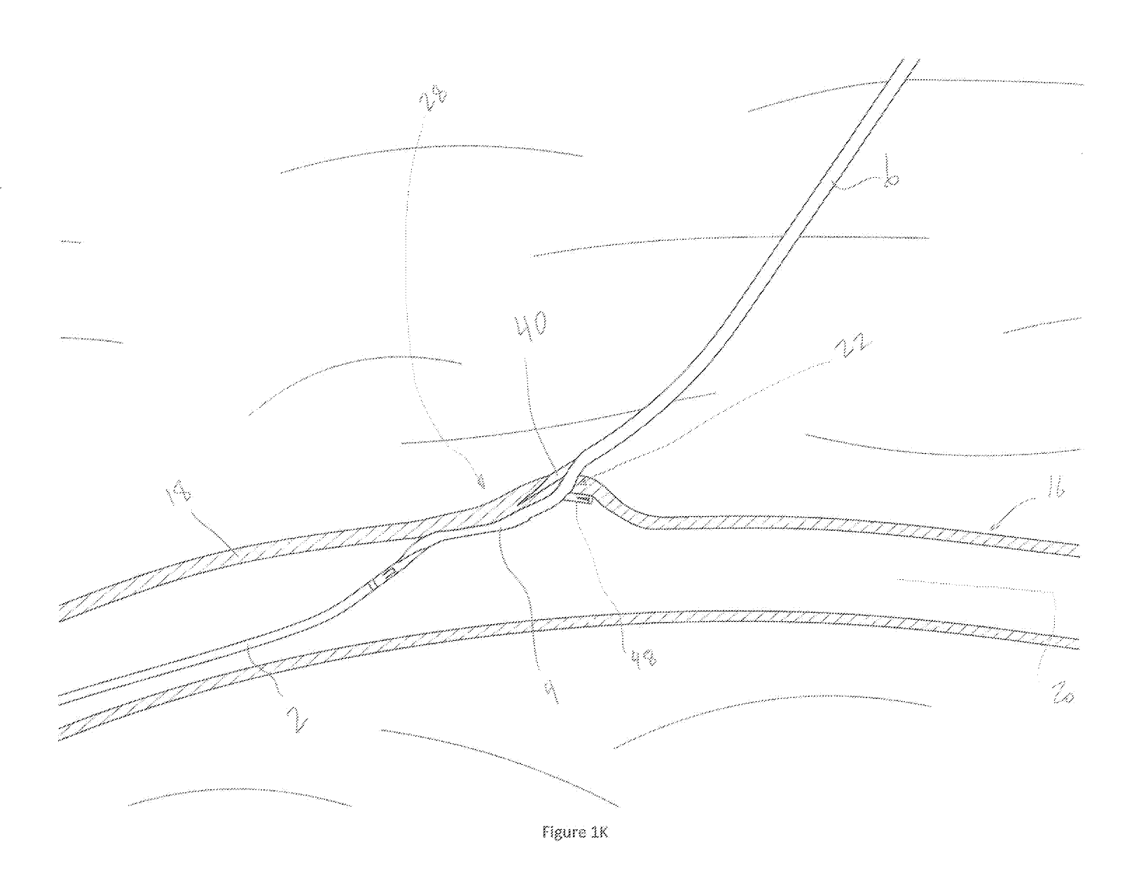

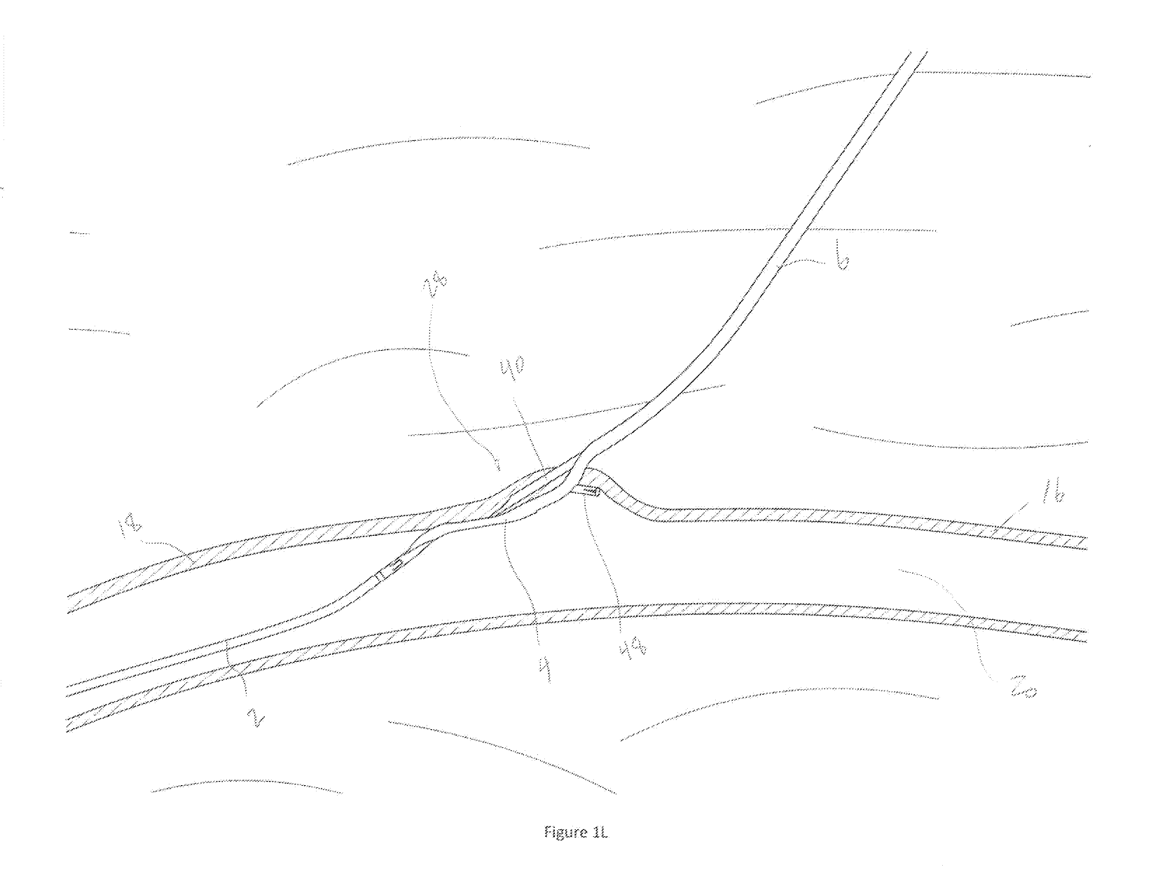

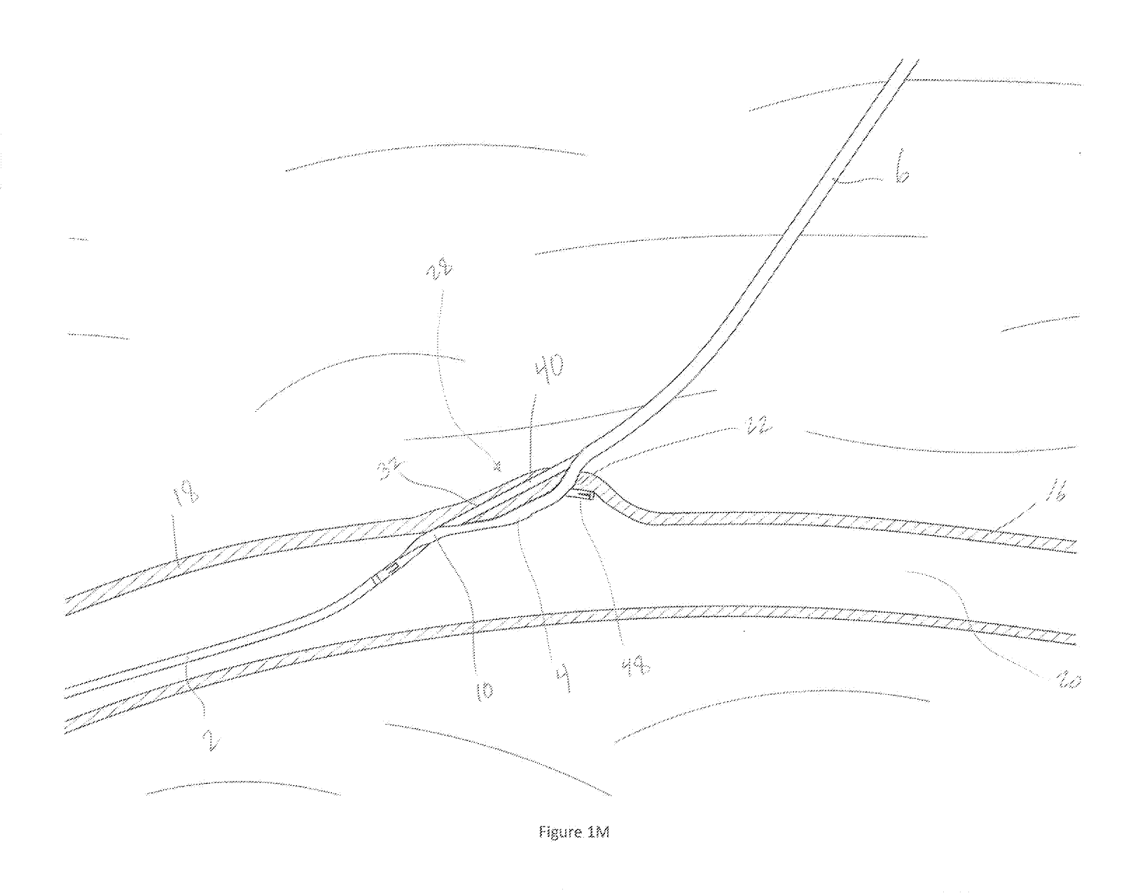

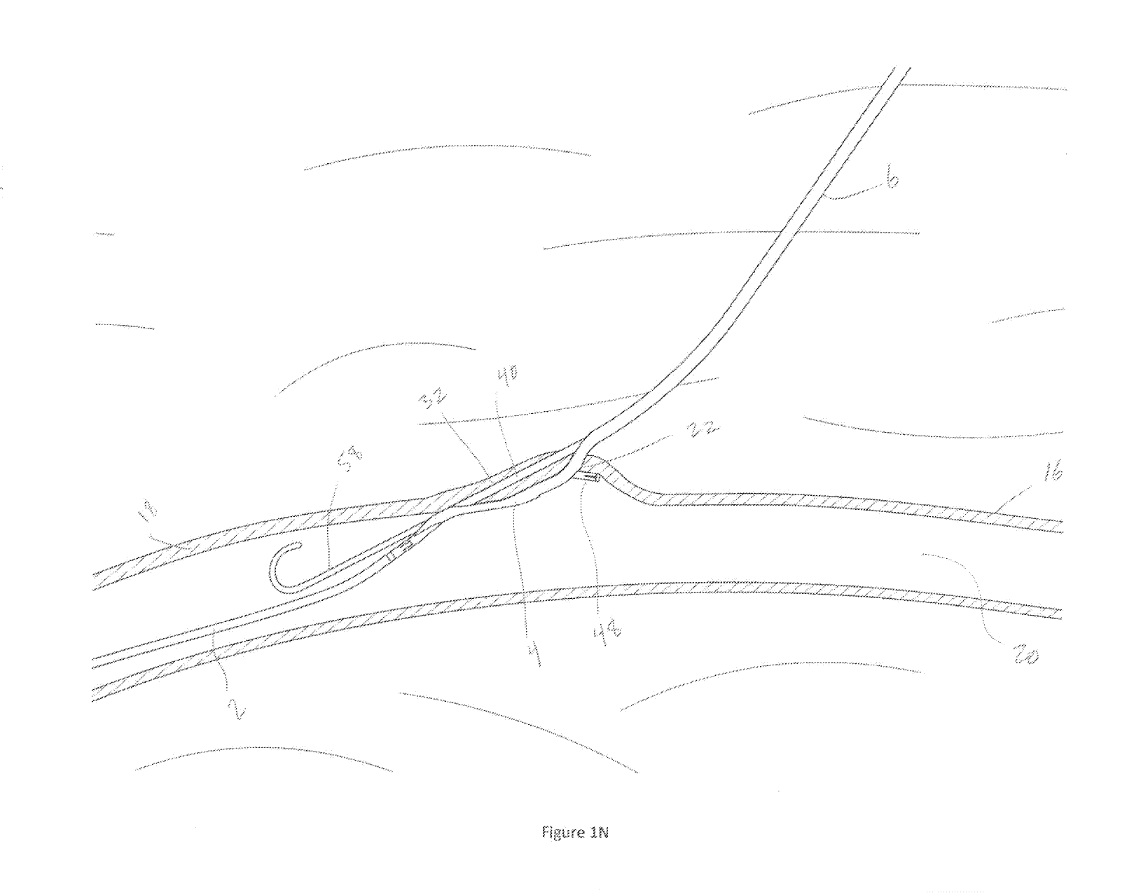

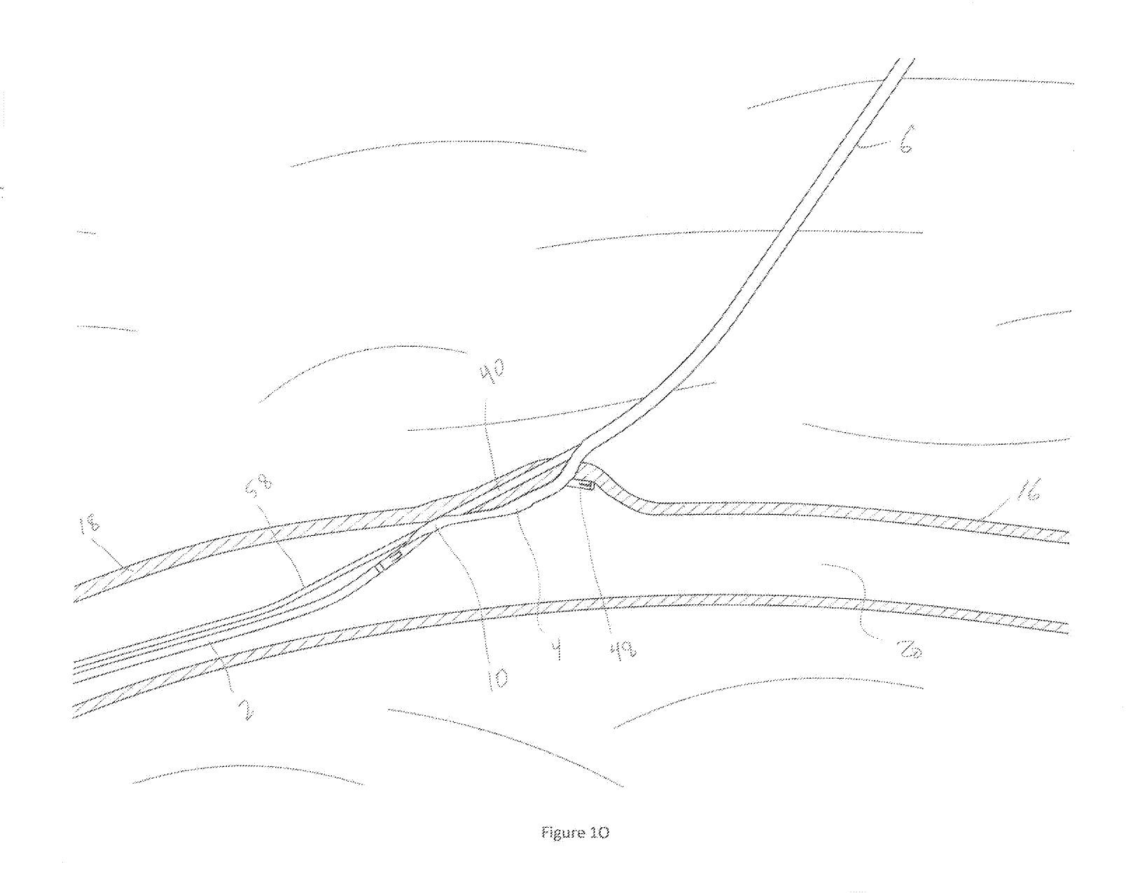

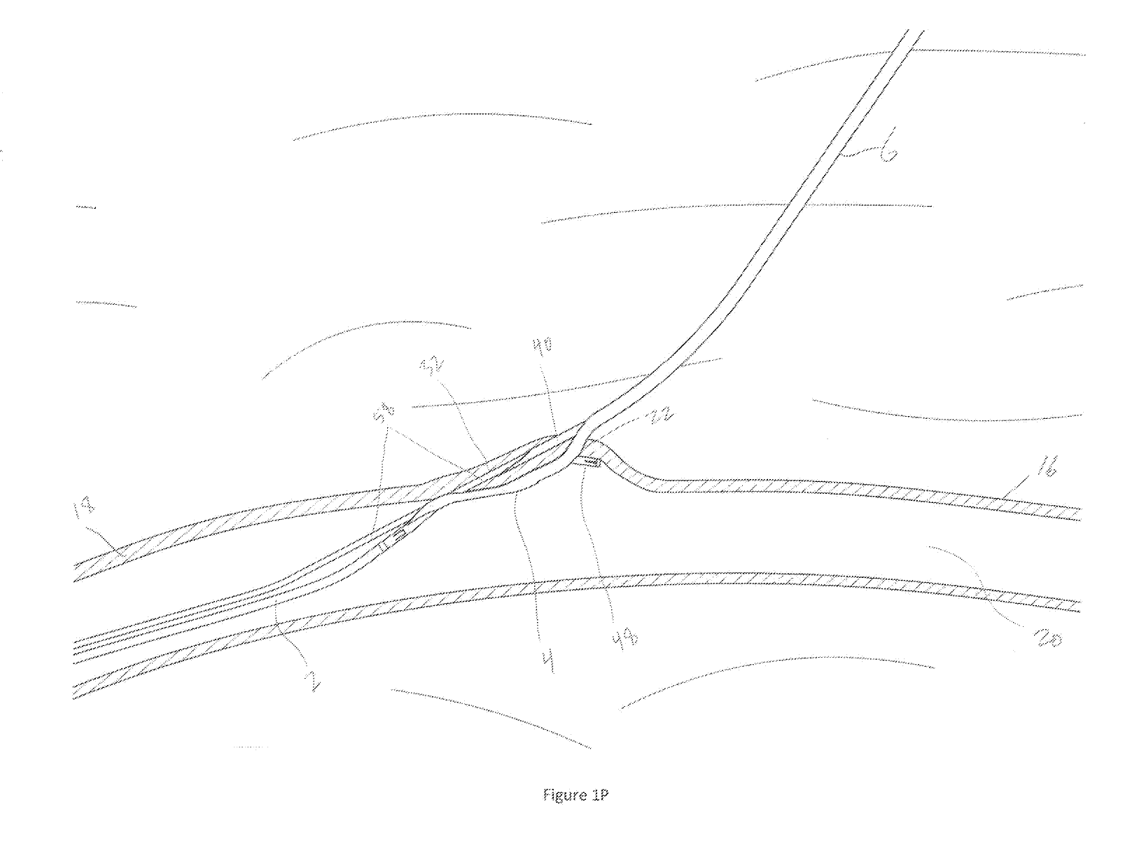

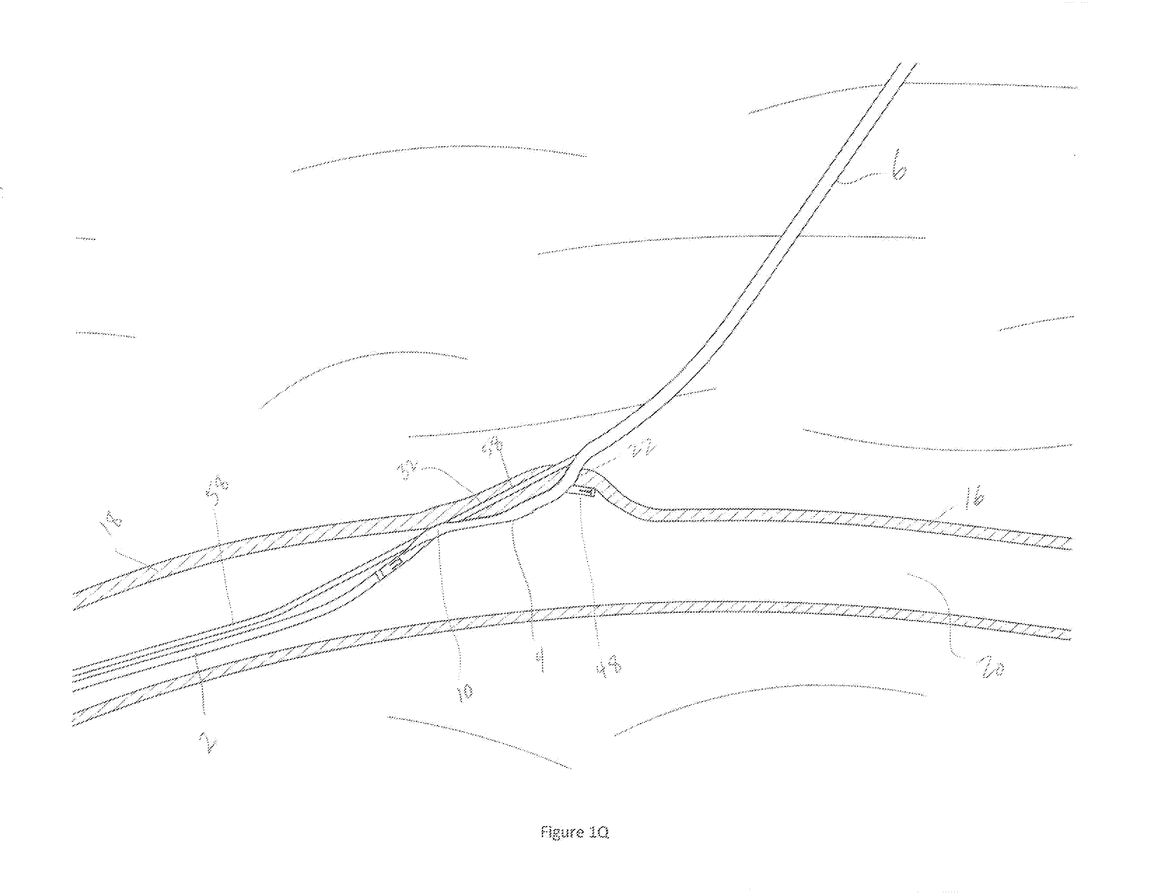

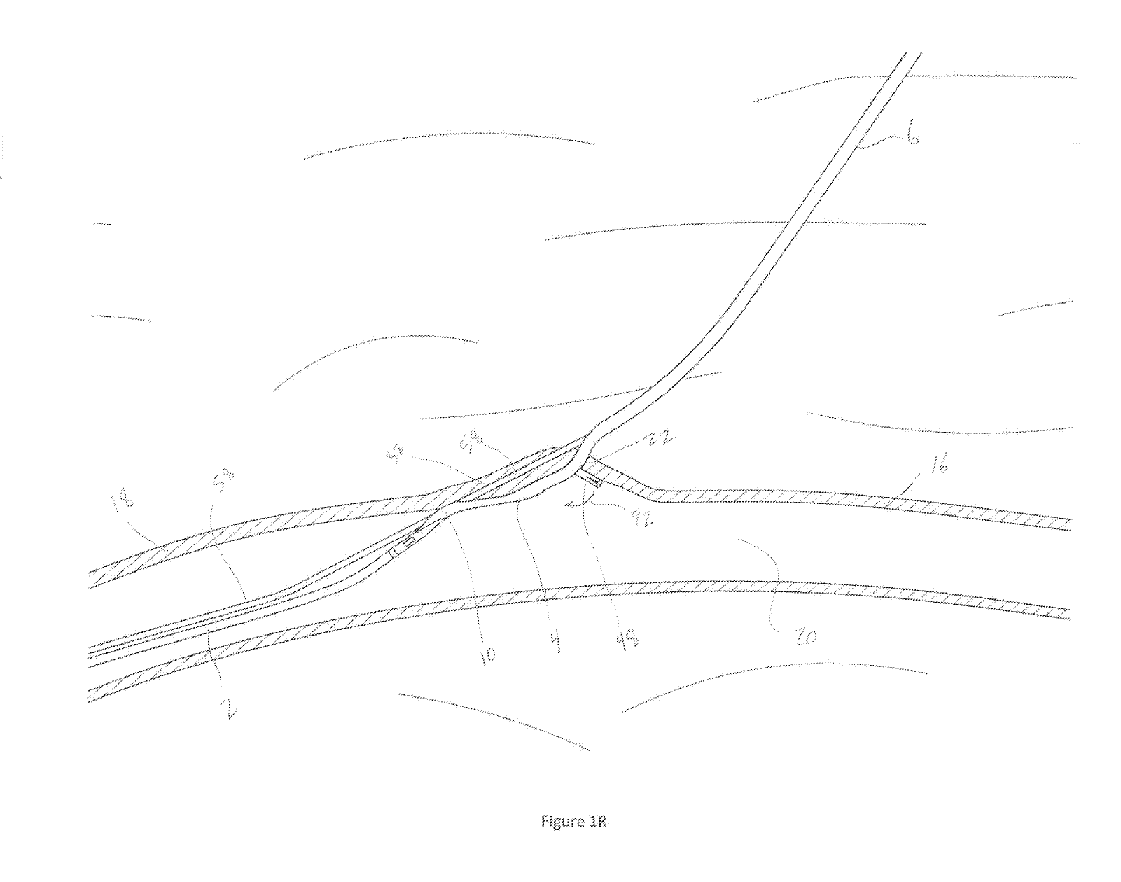

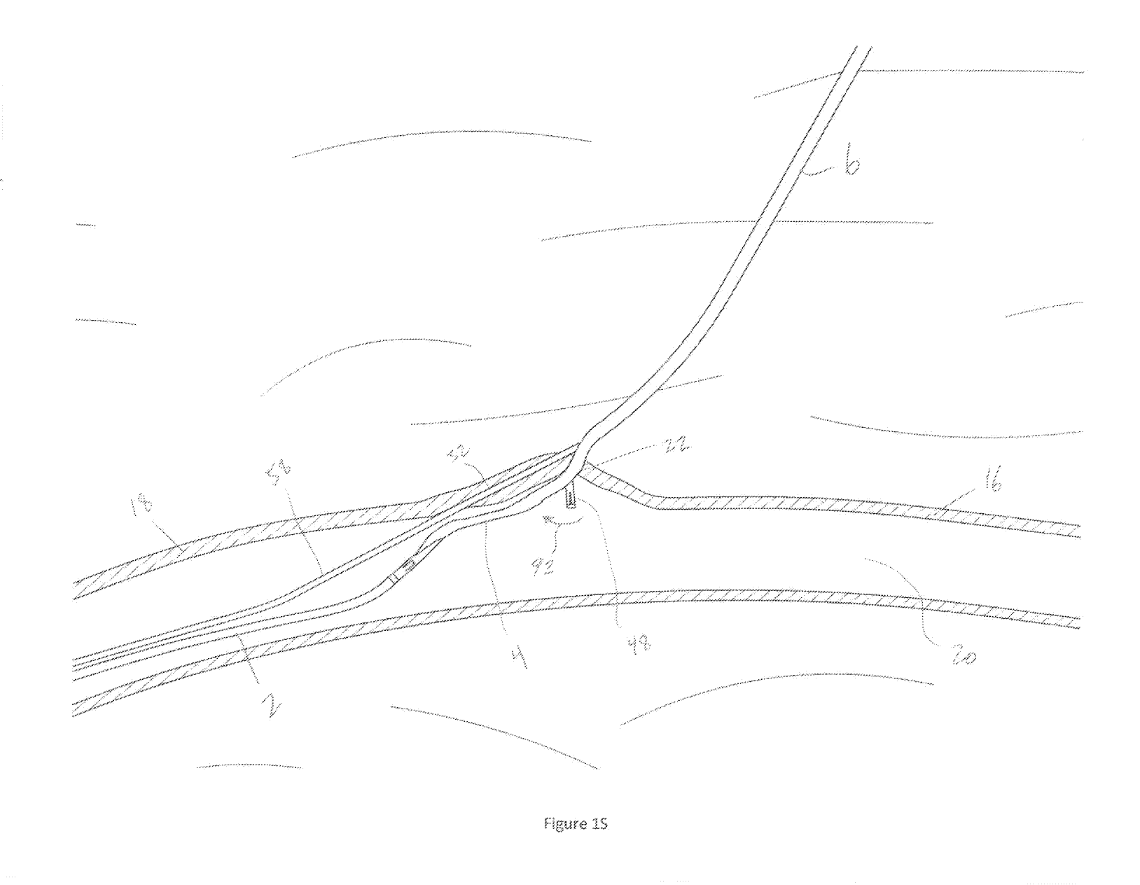

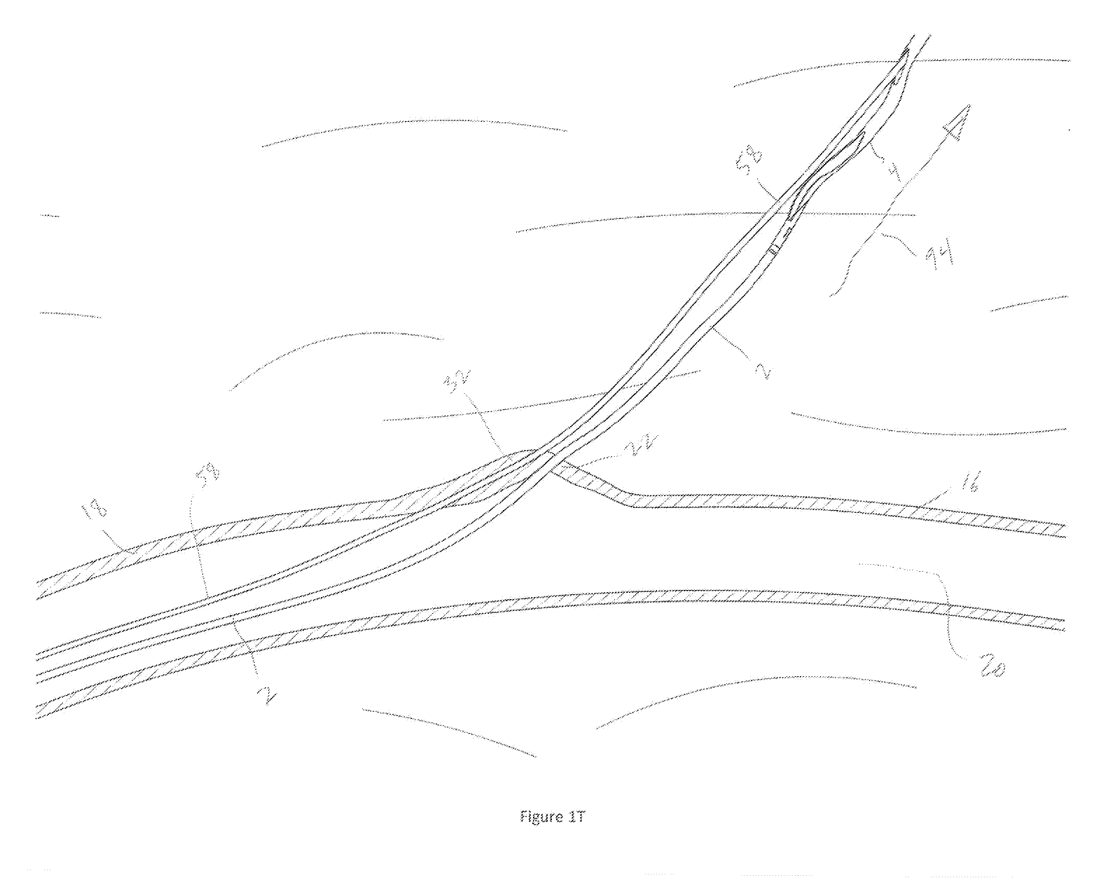

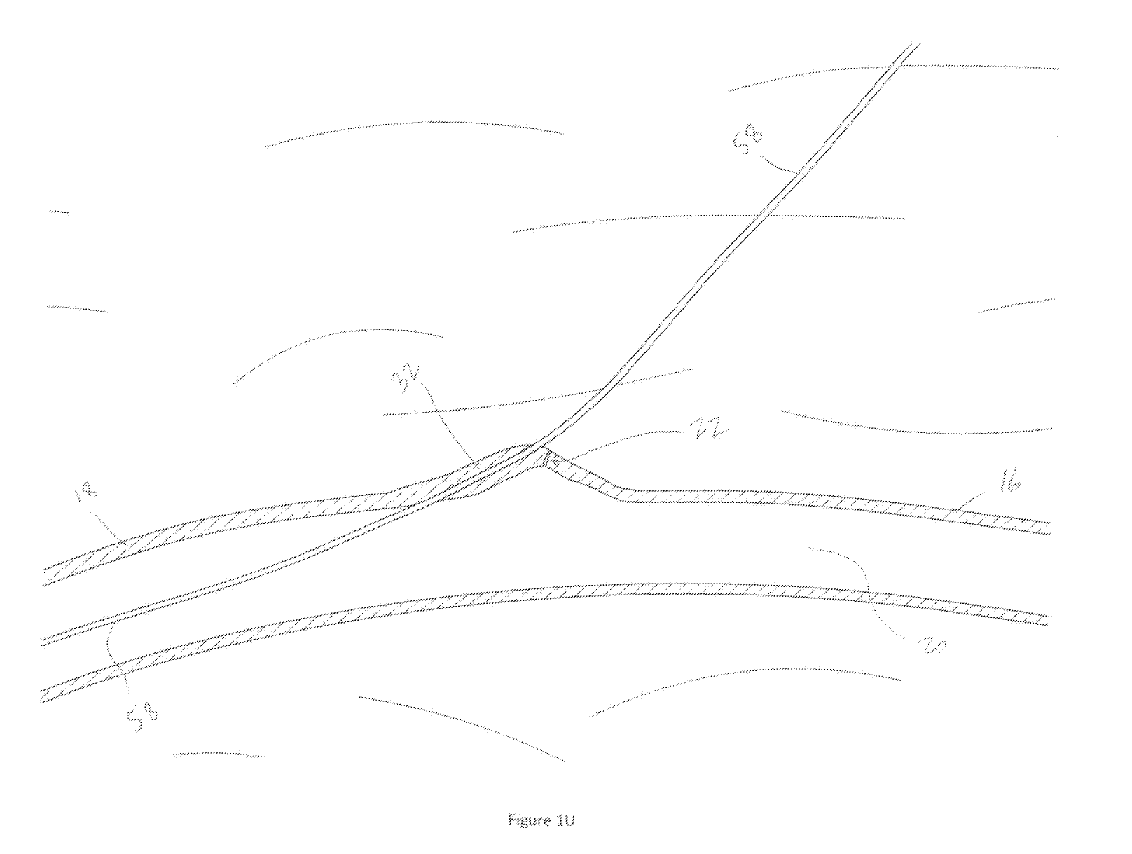

Referring to FIG. 1A, a group of associated tissue structures is illustrated, with a skin layer (12), subdermal tissue layer (14), and blood vessel (16) depicted. The blood vessel (16) is shown in cross sectional form with the vessel walls (18) sectioned and the vascular lumen (20) shown. As shown in FIG. 1A, a flexible distal portion (2) of an anchor assembly has been placed such that a a distal portion (78) resides within the vessel lumen (20) where blood would be flowing, and a proximal end (80) resides external to the patient. In one embodiment, such a flexible elongate member (2) maybe placed using a Seldinger technique, wherein a sharpened object, such as a scalpel, may be utilized to create a small entry point through the skin layer (12), after which a hollow needle may be inserted through the entry point, across the subdermal layers (14), and into the targeted vessel lumen (20) through a first passage (22), the passage generally created by the needle in a Seldinger technique configuration, followed by insertion of the elongate flexible member (2), which may resemble a guidewire in structure and/or function. The proximal aspect (80) of the depicted flexible member (2) comprises a coupling fitting (66) configured to be coupled, or removably coupled, to another portion of an anchor assembly structure, as shown, for example, in FIG. 1B. Referring to FIG. 1B, the coupling fitting (66) of the elongate flexible member (2) is being compressed against a distal portion of the depicted pre-bent midportion (4) of the depicted anchor assembly (the assembly ultimately comprising the elongate flexible member 2, the midportion 4, and the proximal portion 6, which may also feature a proximal manipulation handle 8; a needle member 40 may also be coupled to the assembly, as described below) to result in a coupled assembly, as shown in FIG. 1C.

Referring to FIGS. 1D, 1E, and 1F, with the assembly (2, 4, 6, 8) in a coupled formation, it may be inserted further into the patient in an elongate fashion as shown, with the distal end (78) of the flexible distal portion (2) continuing to insert farther into the vessel lumen (20). Referring to FIG. 1 G, when the proximal aspect of the prebent midportion (4) enters the blood contained within the vascular lumen (20), a small distal port (say in the region of depicted element 87) that is fluidly coupled (i.e., by a “marker channel” or lumen defined through the interconnecting portion of the proximal portion 6 of the assembly) with a proximal blood marker port (86) mounted to the distal end of the handle (8) allows a small flow of blood to travel up the marker channel from the distal port (87) and out (84—the expressed mark of blood itself) of the proximal marker port (86) where it intentionally is viewable by the operator holding the handle (8). In one embodiment, the lumen through which the needle travels in the anchor assembly proximal portion (6) functions as the blood marker channel (i.e., such lumen functions to contain the needle and also to fluidly connect the proximal and distal marker ports). In another embodiment, a specific lumen may be configured to function as a marker channel. Such a blood marking is an indicator that the distal port location (87) of the assembly has reached the vessel lumen (20). Referring to FIG. 1H, with the visible mark of blood (84) proximally through the proximal port (86), the operator may mechanically deploy a load assisting member (48), causing such member to extend away from the assembly midportion (4) and provide a greater net load bearing surface configuration to assist with pullback loading, as described above. Such mechanical deployment may be controlled by intentional controlled movement of a small handle or trigger movably coupled to the proximal handle (8), which may cause a coupled tensile element such as a wire to apply a tensile load upon an aspect of the load assisting member (48), causing it to rotate out (90), as shown in FIG. 1 H, insert out somewhat linearly along an axis, or insert/rotate in a combined or arcuate fashion. In one embodiment, the blood marking described above (a visible mark of blood 84 out of proximal marking port 86) may be utilized to ensure that the load assisting member (48) will be close to the vessel wall (18) when deployed, to prevent undesired relative motion of the load assisting member (48) with other tissue surfaces, such as the opposing vessel wall.

Referring to FIG. 1I, with the load assisting member (48) extended out, a proximal load may be delivered to the assembly through the handle (8) and/or proximal shaft (6) to cause the prebent midportion (4) and load assisting member (48) to urge the nearby portion of the vascular wall (18) into a specific desired contact configuration (28). In one embodiment, in a desired contact configuration (28), the nearby portion of the vascular wall is folded around and urged against the prebent midportion, somewhat akin to the manner in which a towel hangs over a horizontal towel rod in a bathroom due to gravity-based loading. FIG. 1J shows a closer view of the desired contact configuration (28) featured in FIG. 1I. Referring to FIG. 1K, the importance of the shape of the prebent midportion and the desired contact configuration (28) are emphasized with insertion of a needle member (40) across the adjacent vessel wall portion (18) to create an expandable tract. In the depicted embodiment, the needle member (40) is movably coupled through a port and lumen defined through the proximal member (6) which leads to a plunger (96) that is movably coupled to the handle (8), enabling an operator to move the needle in insertion and retraction while also retaining the desired contact configuration (28). FIG. 1L shows further insertion of the needle member (40). FIG. 1M shows full insertion of the needle member (40) into a configuration wherein the distal portion of the needle is placed into contact with a saddle-shaped surface of the prebent midportion (4) which serves to constrain motion of the needle (40). Indeed, in one embodiment, the prebent midportion (4) is specifically designed to have a canoe-like geometry configured to guide the needle (40) tip through a valley-like spine of the distal aspect of the prebent midportion (4) and into the configuration shown in FIG. 1M wherein the needle (40) tip is safely and predictably motion-constrained. With the cutting action of the needle member (40) complete and the expandable tract (32) created through two now overlapping portions of the vessel wall (18; i.e., bisected by the expandable tract 32 and oriented at an angle relative to a longitudinal axis of the vessel wall or vessel lumen itself), another elongate flexible member, such as a guidewire (58), may be inserted through a lumen formed through the needle (40), as shown in FIG. 1N. Referring to FIG. 1O, more length of the guidewire (58) has been inserted with the needle (40) remaining in place movably coupled to the anchor assembly.

Referring to FIG. 1P, with adequate desired length of the guidewire (58) inserted into the vascular lumen (20), the needle member (40) may be retracted (i.e., by using the movable needle insertion/retraction aspect of the proximal handle 8), leaving the guidewire (58) and anchor assembly (2, 4, 6) in place. The load assisting member (48) may be kept in position to assist with mechanical stabilization during this phase. FIG. 1Q depicts further retraction of the needle member proximally into the proximal member (6) of the anchor assembly. At this stage, an expandable tract (32) has been carefully and safely created through the wall (18) of the vessel (16) in a configuration wherein, as described below, it preferably will self-seal when instrumentation is removed from the tract—and a guidewire (58) has been left in place through the expandable tract (32). The load assisting member (48) may be moved (92) back into a non-deployed position by applying or releasing a load proximally at the handle (8). Further movement (92) of the load assisting member (48) is illustrated in FIG. 1S. With the load assisting member (48) either retracted fully into the body of the anchor assembly midportion (4), or left in a configuration wherein it will not appreciably cause additional resistance to proximal pullout of the assembly, the assembly may be pulled out (94), as shown in FIG. 1T, leaving behind a configuration such as that shown in FIG. 1U, wherein the guidewire (58) remains positioned through the expandable tract (32), and wherein the relatively small initial first passage (22) is closed, preferably by natural hemostasis due to the relatively small size of the first passage (22) that is required to deploy the subject instrumentation.

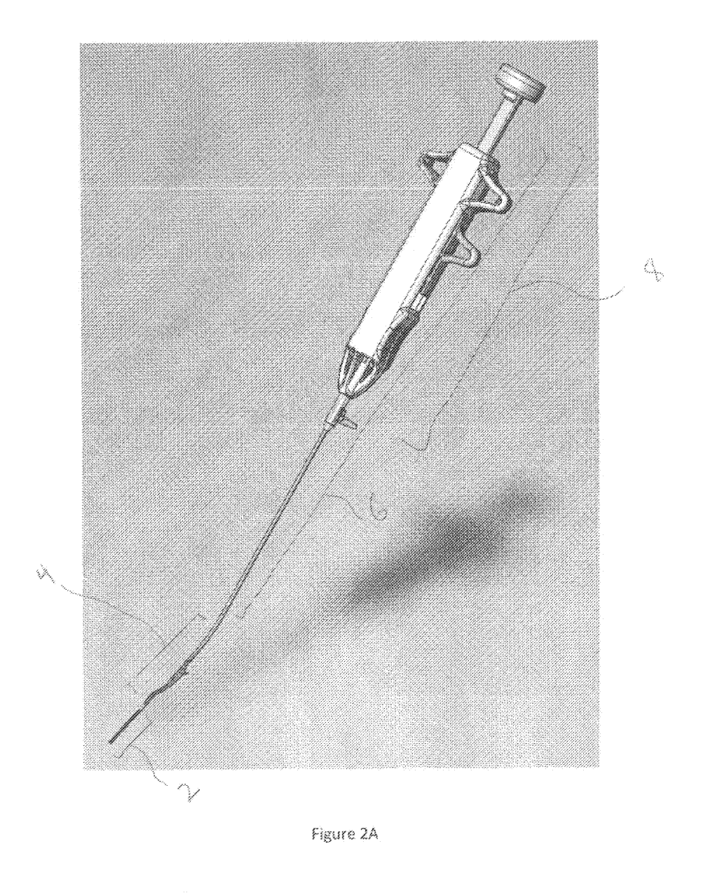

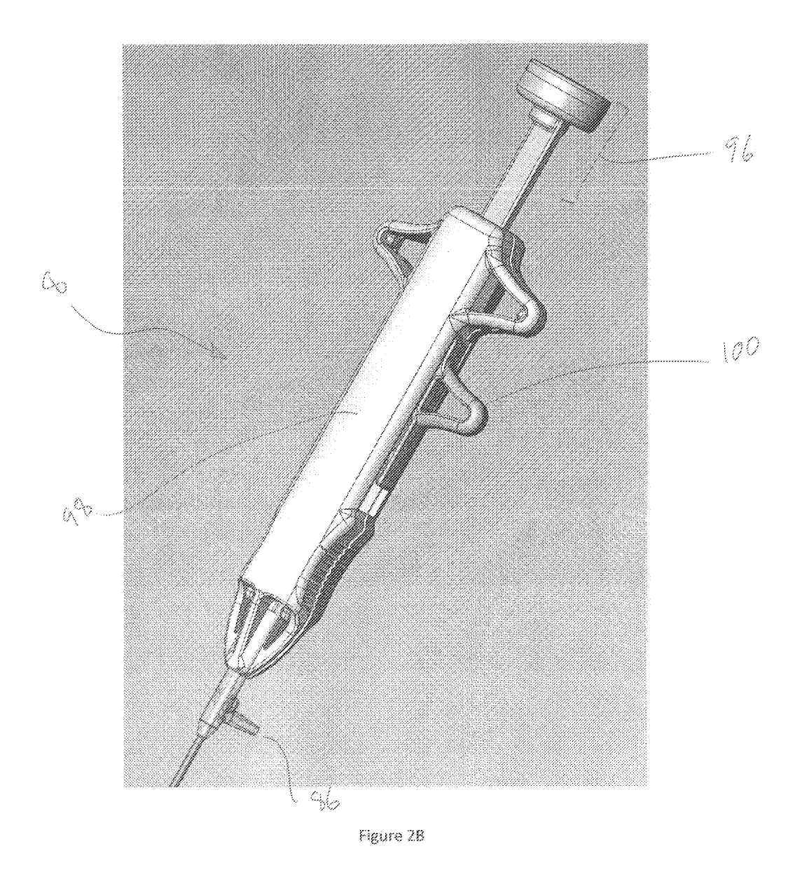

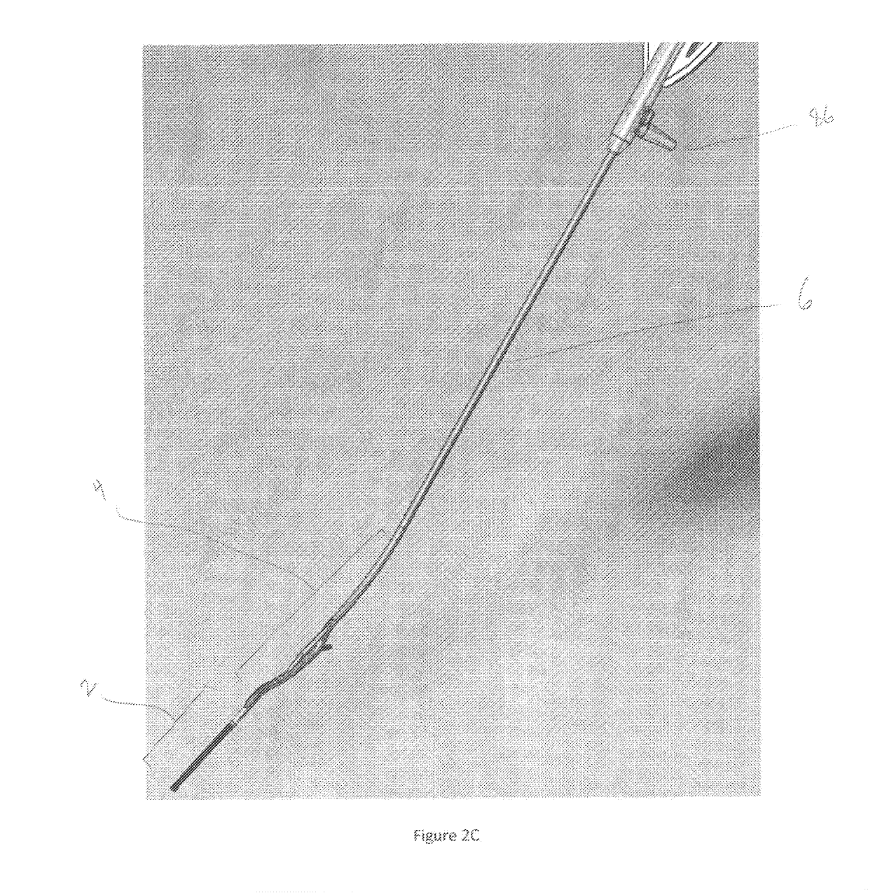

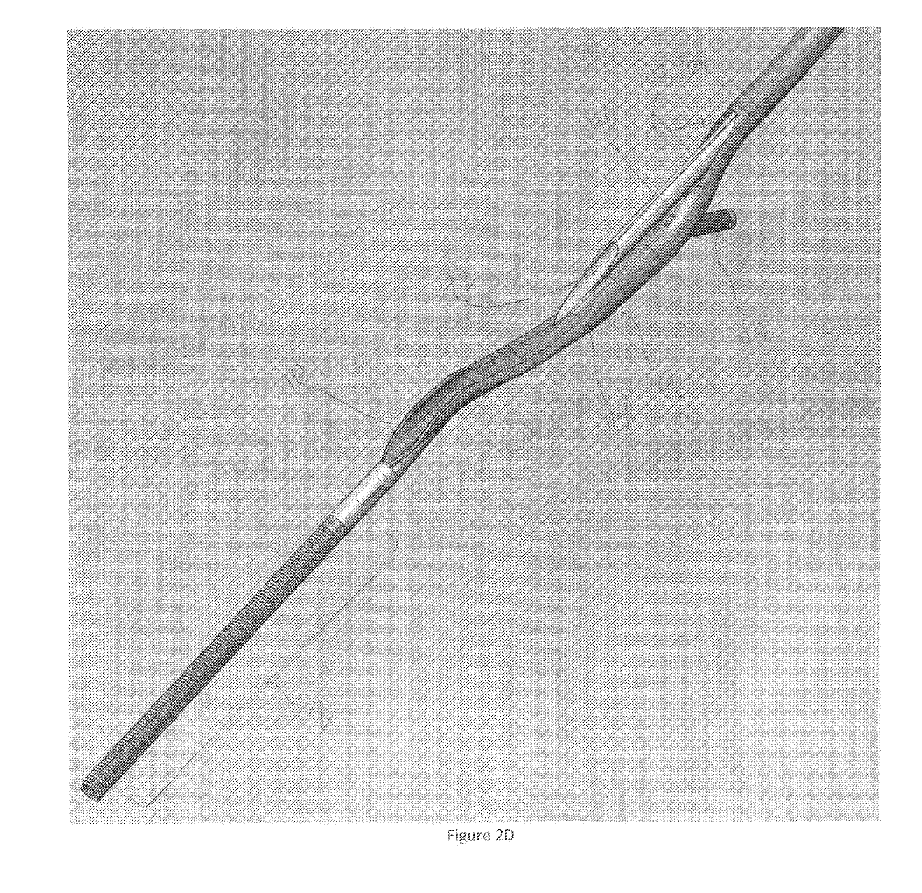

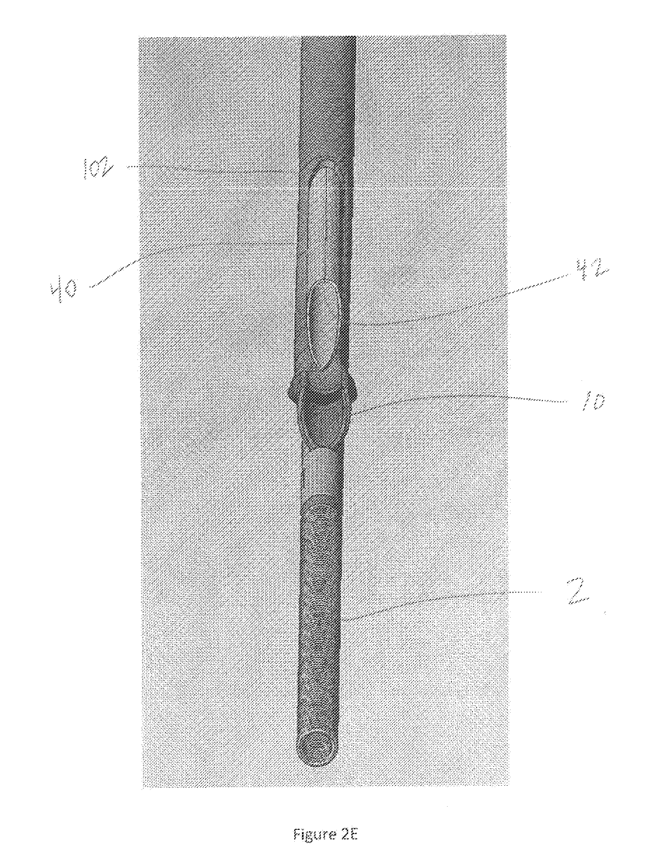

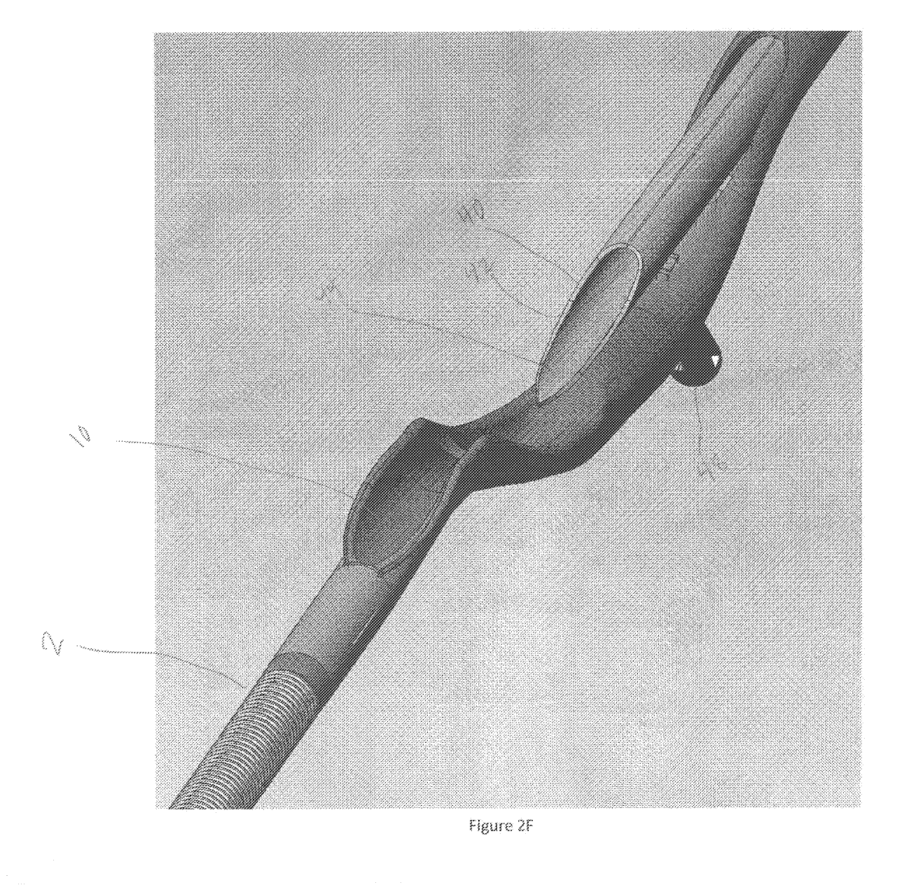

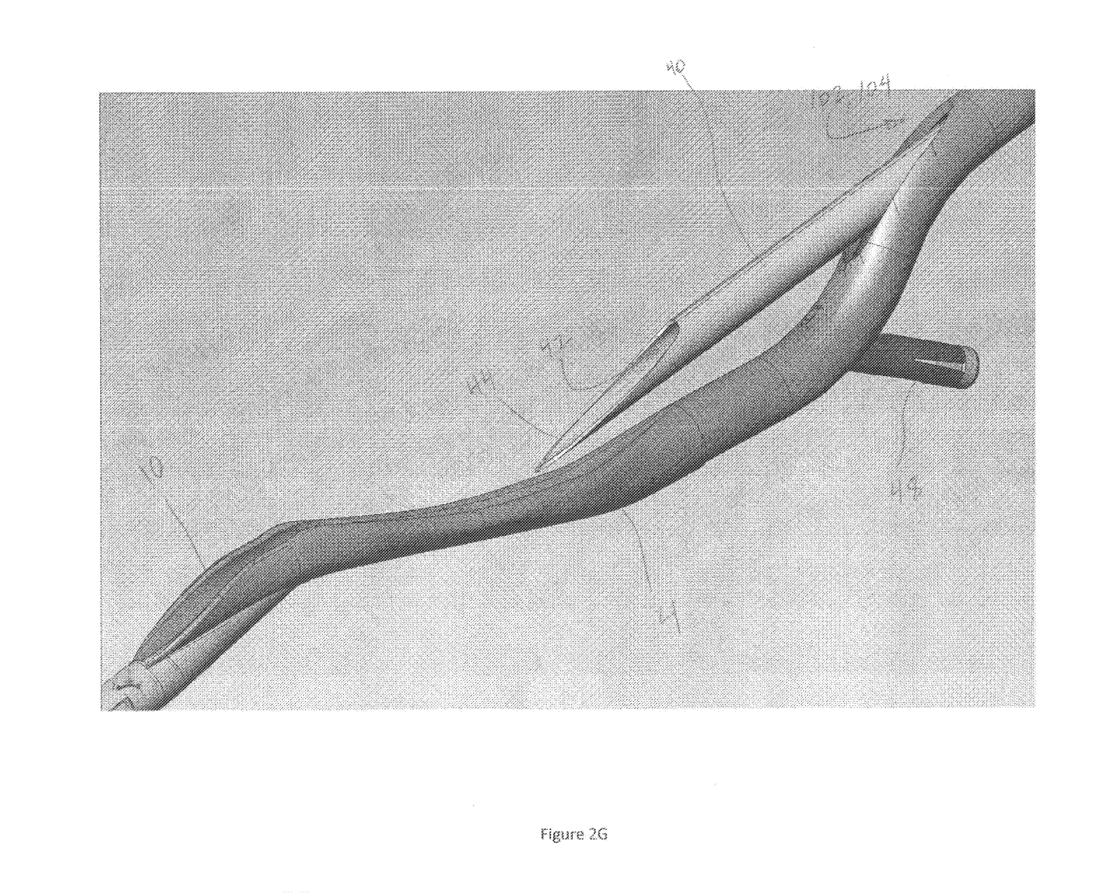

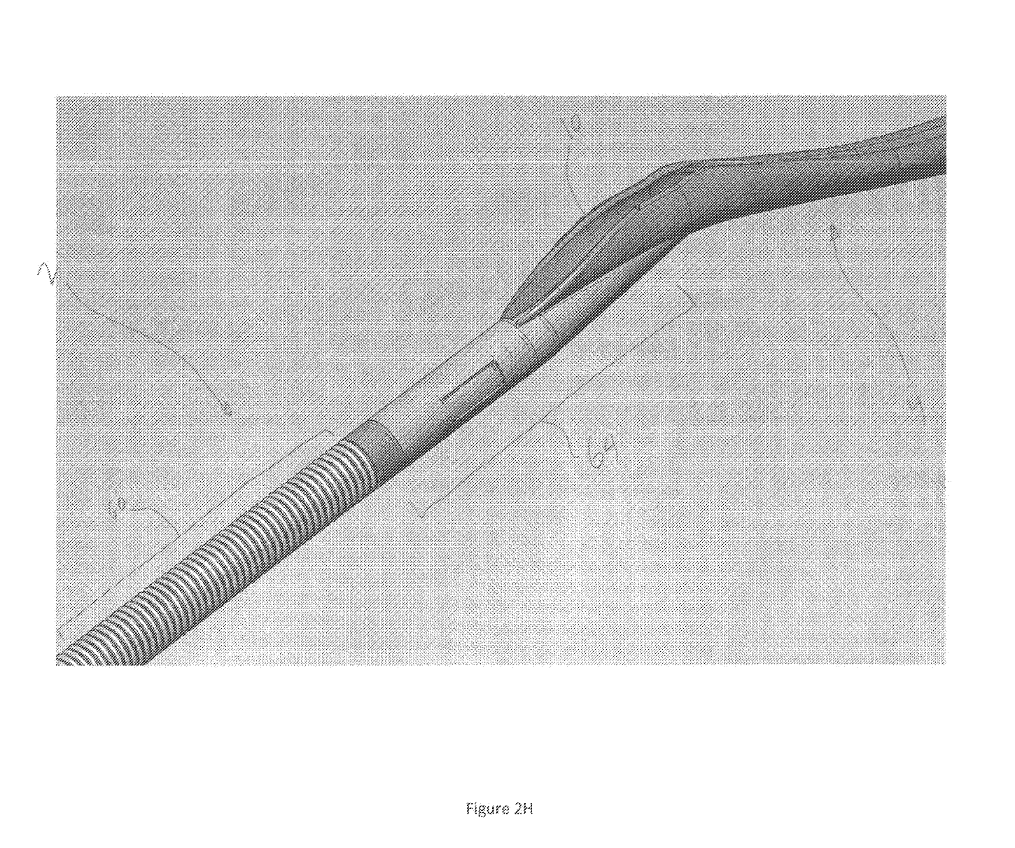

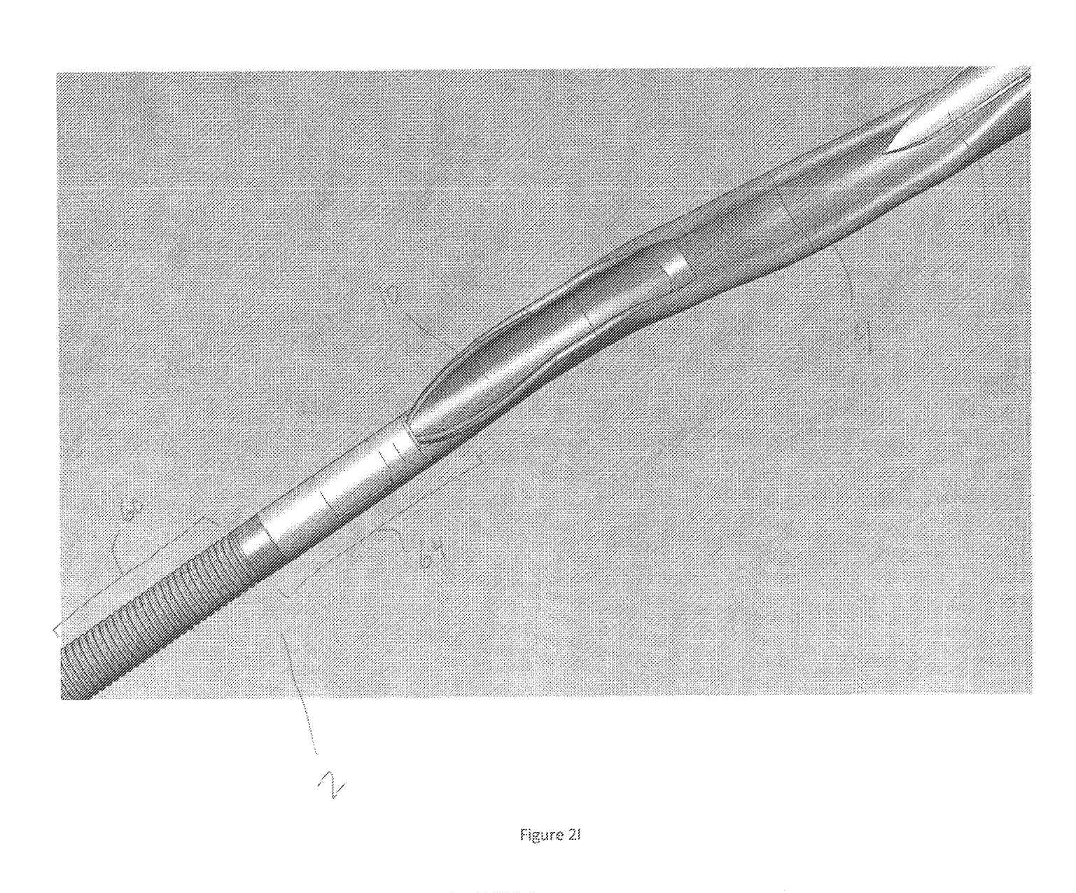

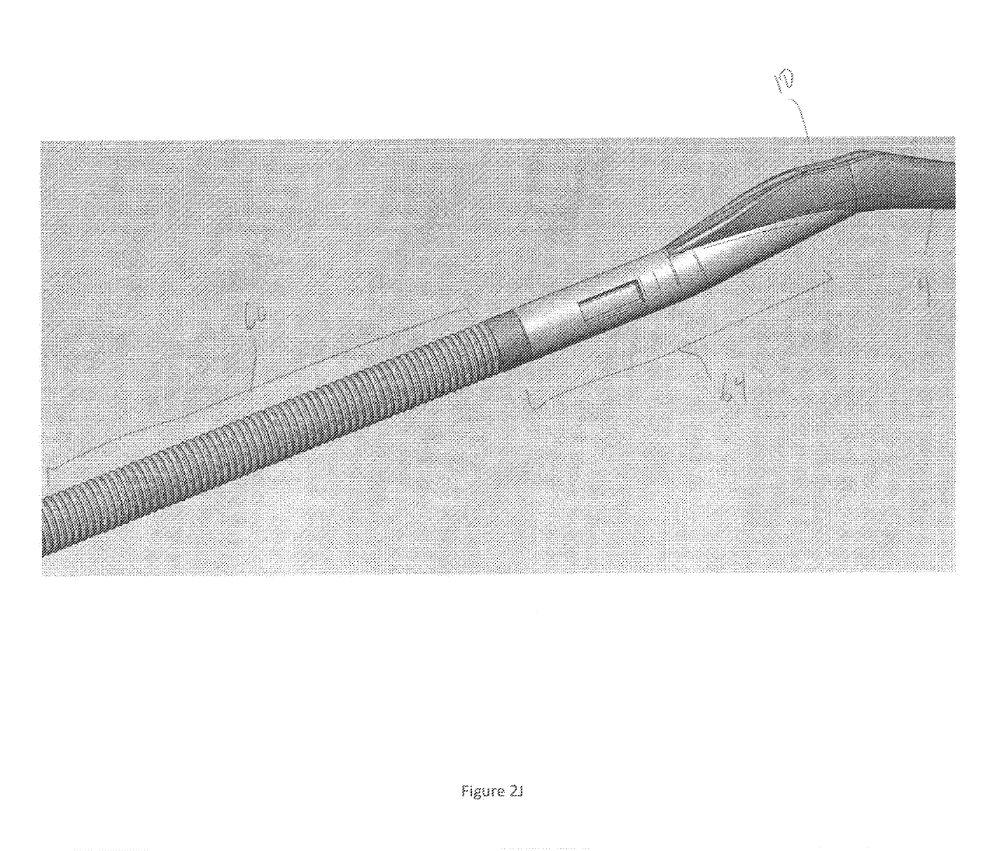

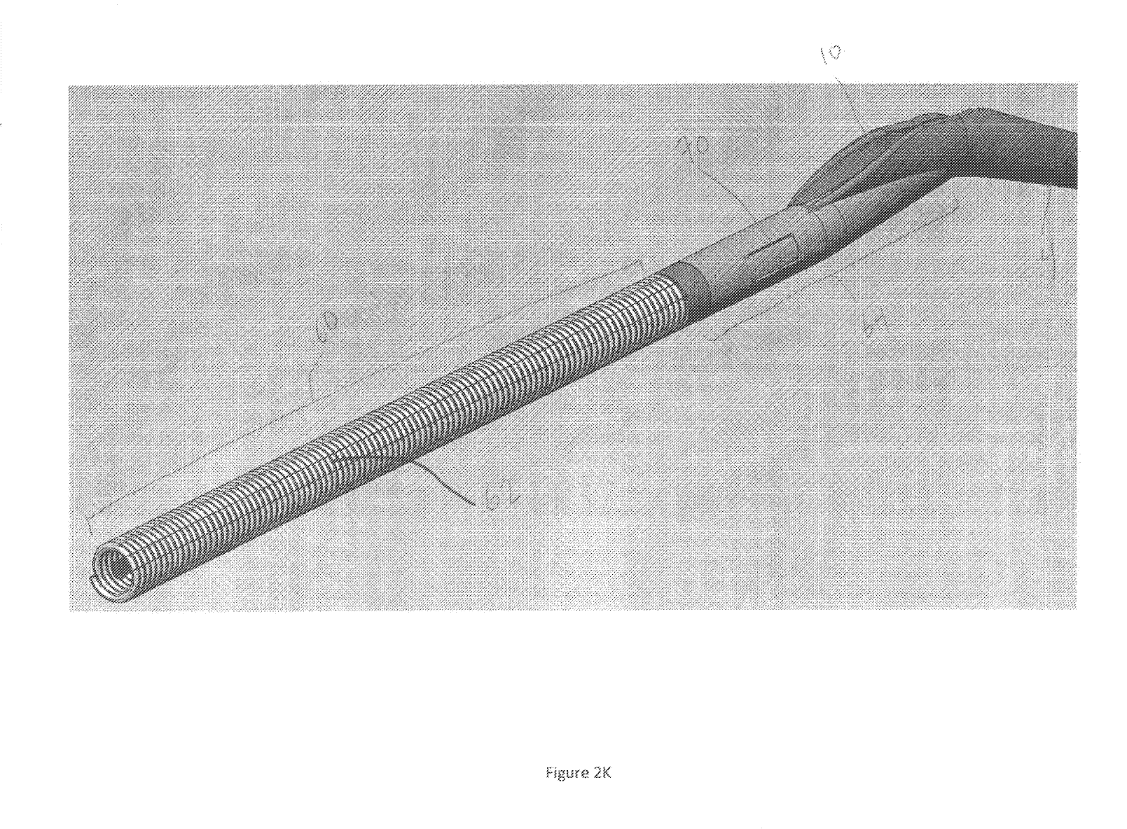

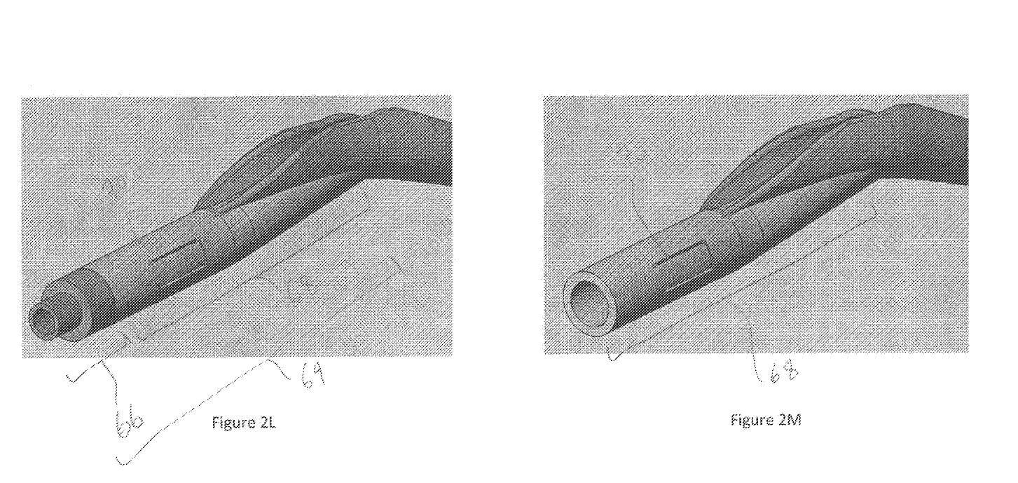

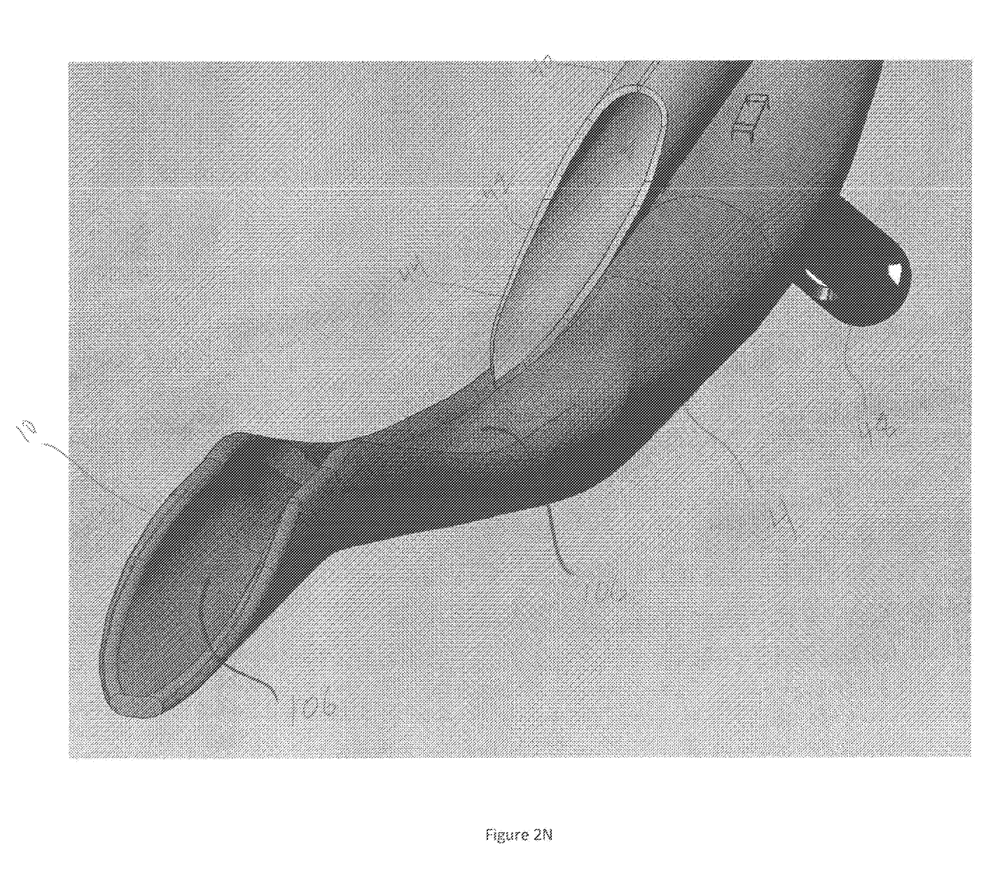

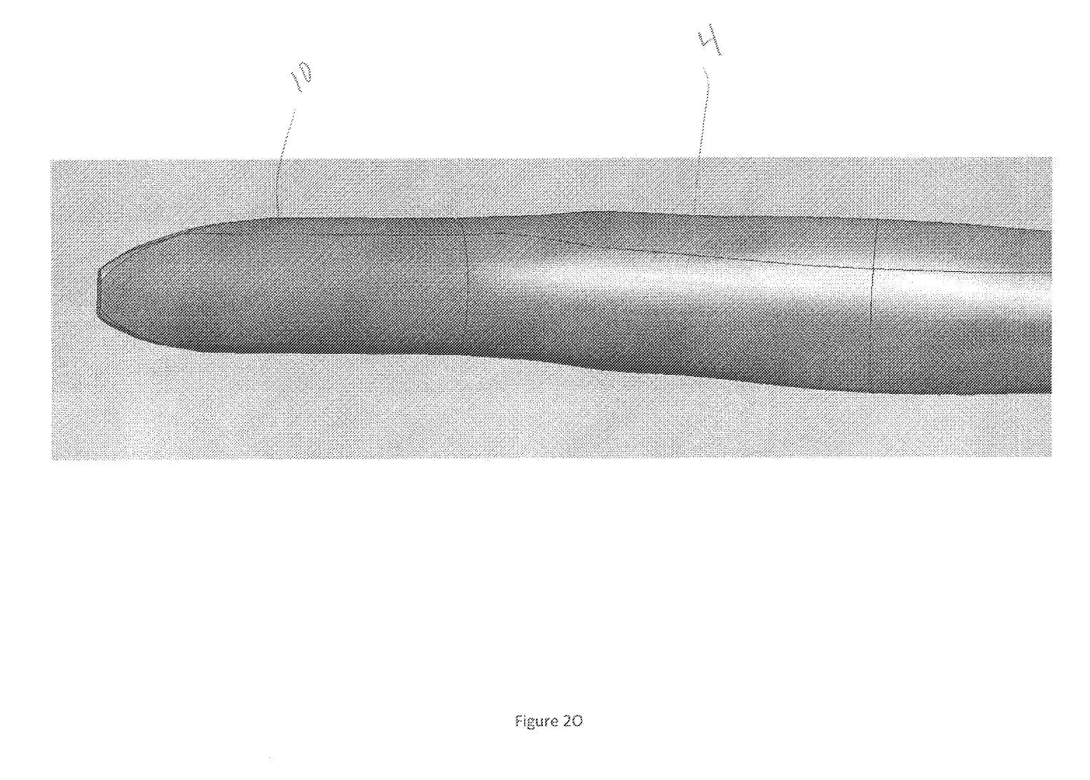

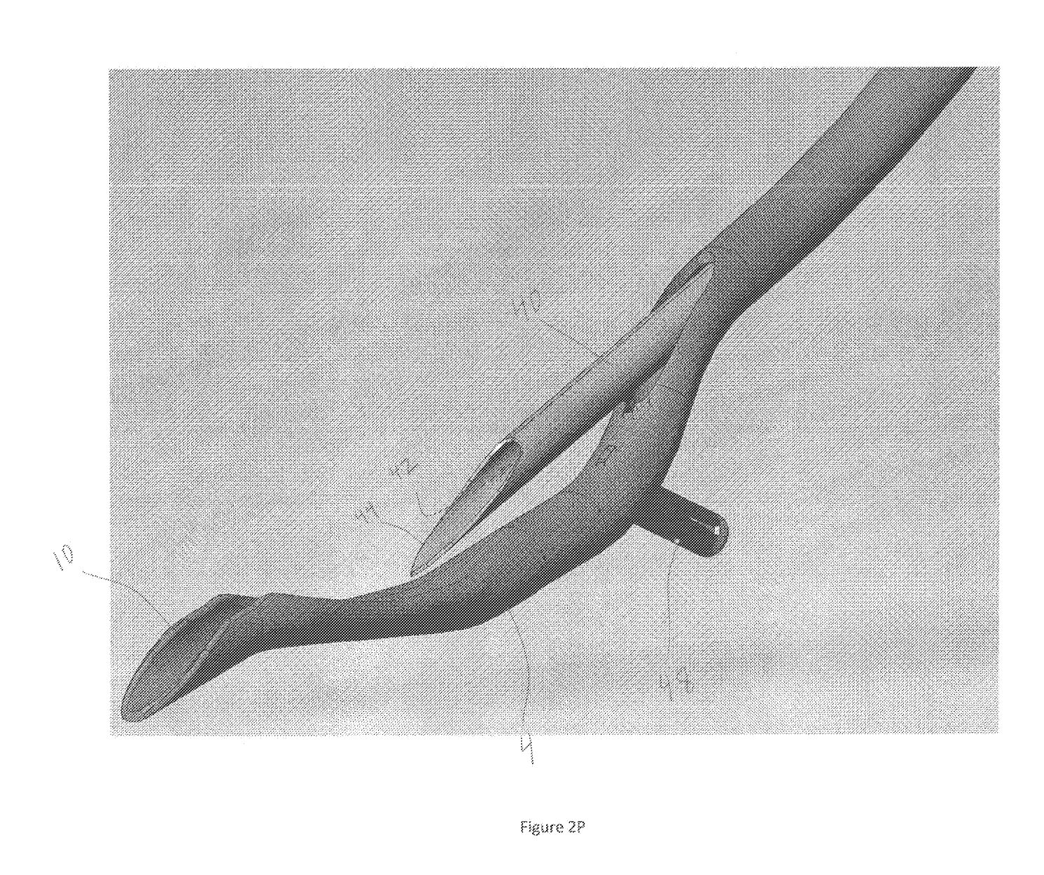

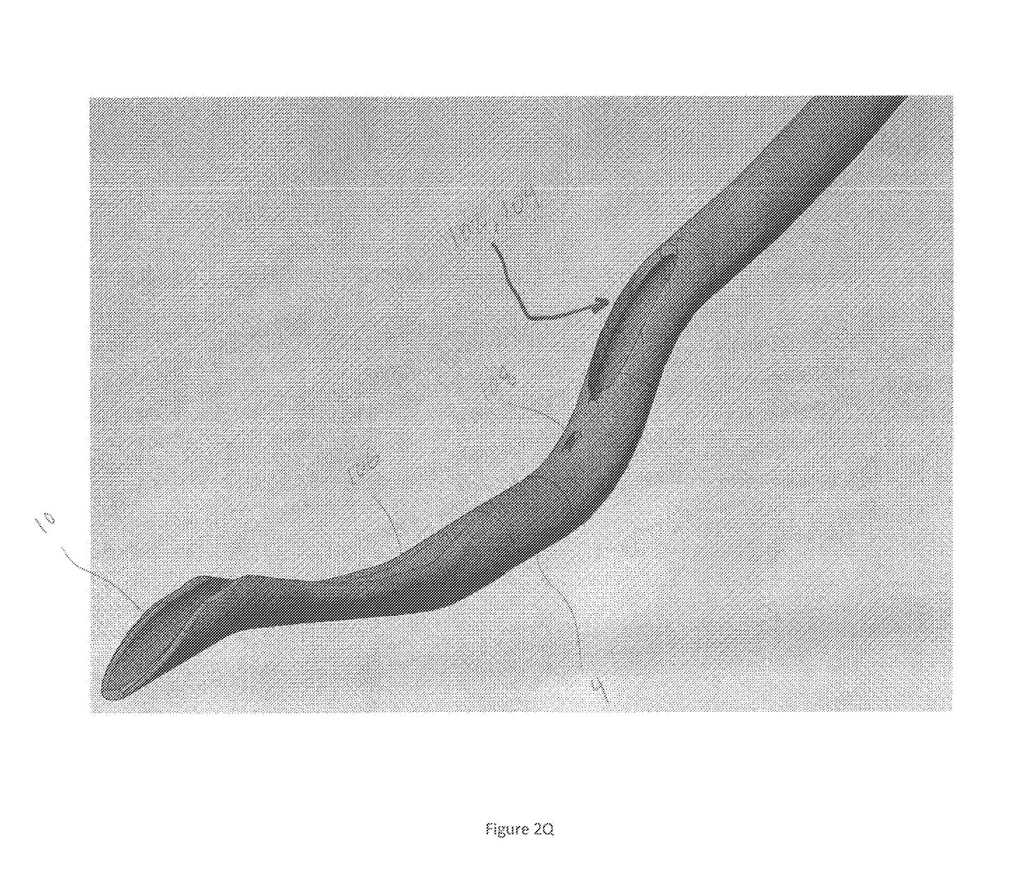

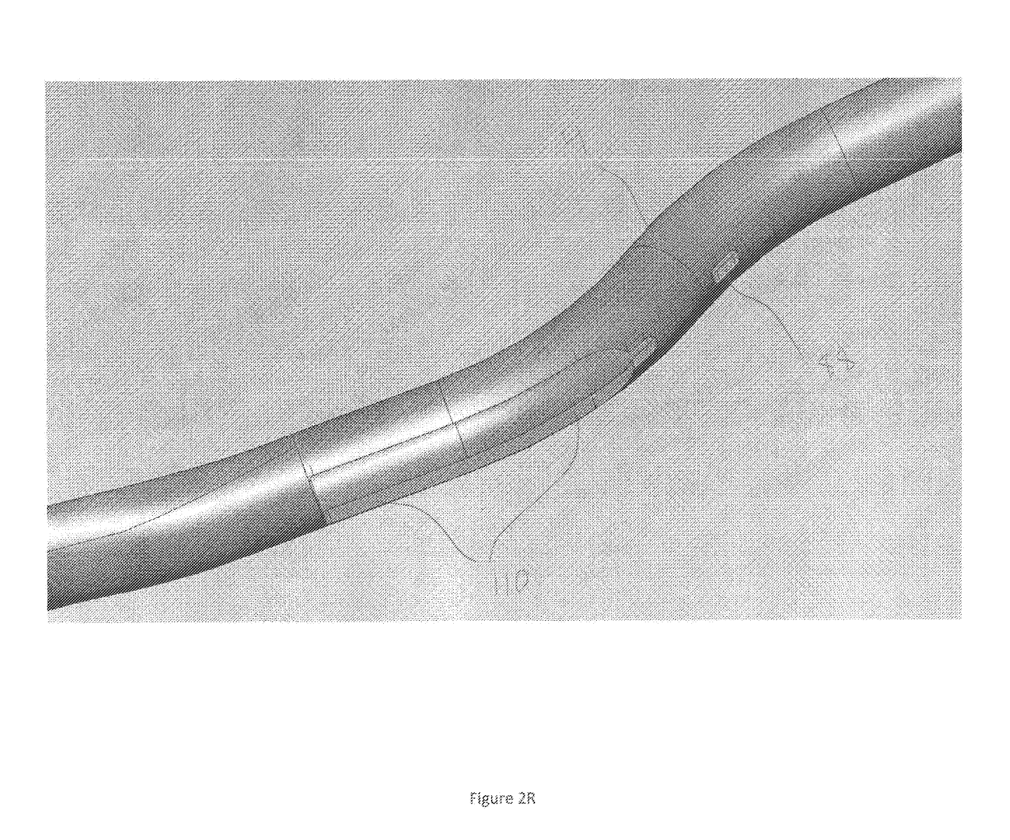

Referring to FIGS. 2A-2T, three dimensional illustrations are depicted to assist with visualizing various aspects of embodiments of the invention. Referring to FIG. 2A, an assembly is depicted comprising a flexible distal anchor portion (2) coupled to a prebent anchor midportion (4), coupled to a proximal portion (6) comprising a handle assembly (8). FIG. 2B shows a closer view of the handle assembly (8), featuring a needle plunger (96) movably coupled to a main housing (98), the needle plunger coupled to a needle such as those described above (element 40). A deployment member (100) also is movably coupled to the main housing (98), and is coupled, in one embodiment via a tension element, to a load assisting member (48) which may be controllably deployed away from a portion of an anchor assembly, as described above. Also shown in the close up view of FIG. 2B is a proximal mark port (86) which may be utilized to assist and operator in positioning an anchoring assembly relative to a vascular lumen, also as described above. FIG. 2C illustrates a close-up view of portions of an anchor assembly, including a flexible distal portion (2), a prebent midportion (4), here movably coupled to a hollow needle (40), and an elongate proximal portion (6). FIG. 2D illustrates yet a closer view of portions of an anchor assembly and associated movable needle. Referring to FIG. 2D, a flexible distal portion (2) may comprise a coiled wire construct. The distal aspect of the prebent midportion (4) may comprise a saddle-shaped needle receiving structure (10), which, as described above, may be configured to provide a mechanically constraining geometry for a needle (40) to slide into. In one embodiment, about ½ of the outer surface of the associated needle (40) local to the receiving structure (10) may be physically constrained via a movable association with the receiving structure (10) geometry. The view of FIG. 2D illustrates that in one embodiment, a needle (40) may be movably coupled through a port (104) and associated lumen (102) defined through a portion of the anchor assembly midportion (4) or proximal portion (6). FIG. 2E depicts an alternate view of similar structures. FIG. 2F depicts a further closer view of an anchor assembly and associated needle (40) with lumen (42) defined therethrough to accommodate a guidewire or other elongate structure. The needle (40) tip (44) may comprise various cutting geometries, such as a trocar tip geometry, chiseled tip geometry, or scoop-like geometry. An alternative perspective view is depicted in FIG. 2G, which includes a close side-view of a load-assisting member (48) deployed away from the pre-bent anchor midportion (4) to which it is movably coupled.

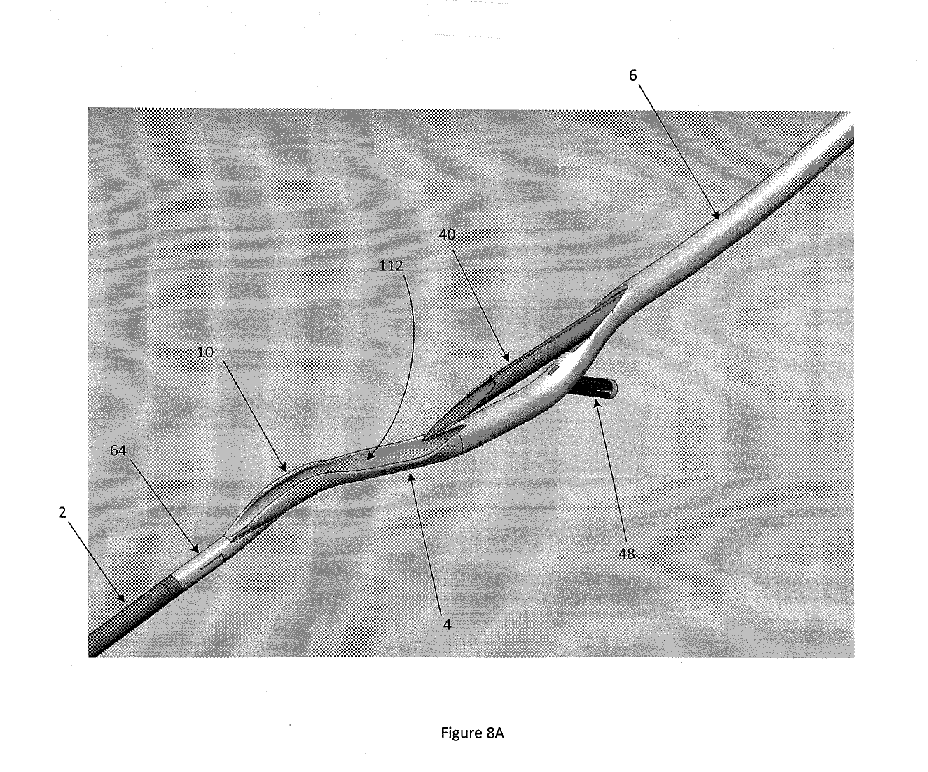

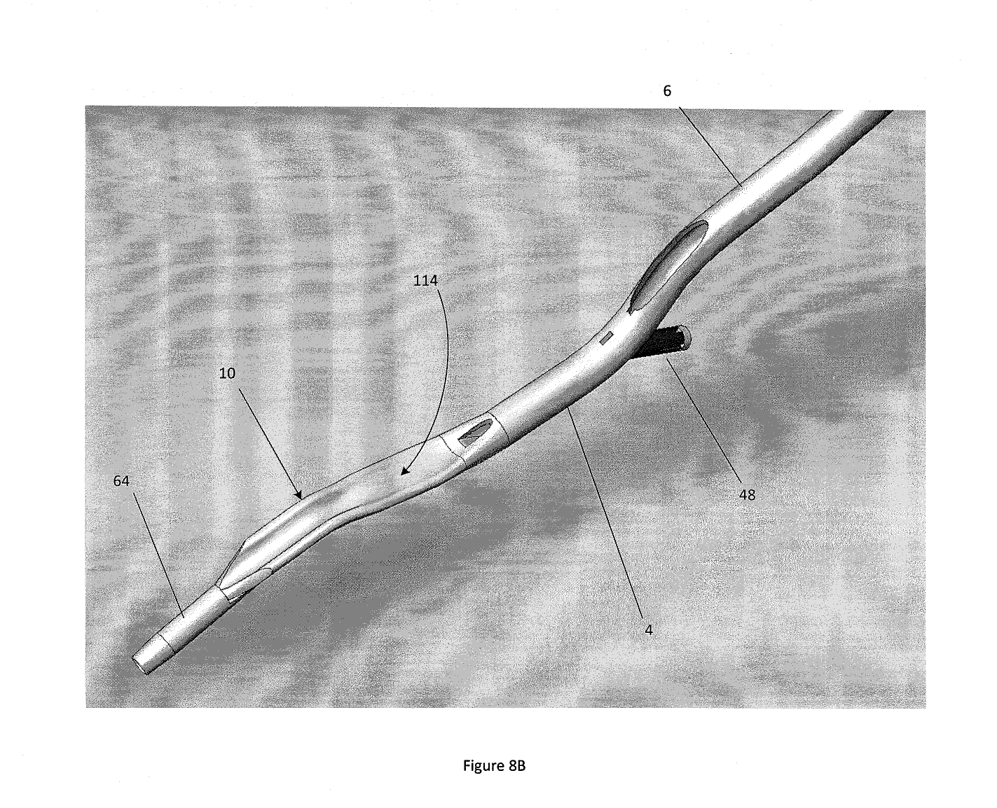

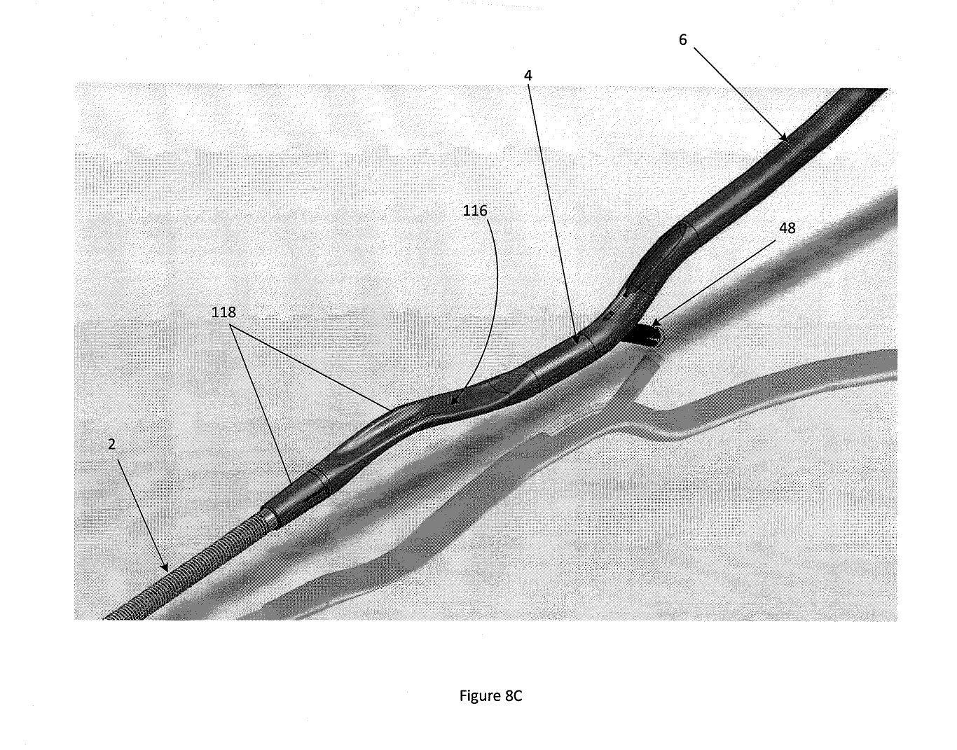

Referring to FIG. 2H, a flexible distal anchor portion (2) may comprise a coiled wire portion (60) coupled to a saddle-shaped needle receiving structure (10) of a pre-bent anchor midportion (4) by a junction assembly (64) which, as shown in FIGS. 2L and 2M, may comprise a first coupling member (66) coupled to a coiled wire portion (60) and a structural core wire (element 62 in FIG. 2K). The first coupling member (66) may be removably coupled to a second coupling member (68) using one or more latch features (70), such as the cantilevered tab (70) formed into a portion of the second coupling member (68) as shown in FIG. 2M. FIGS. 2I and 2K shown differing perspective close-up views to illustrate the shape of the saddle-shaped needle receiving structure (10) formed into the distal end of the pre-bent anchor midportion (4), which may be welded or glued to the junction assembly (64). As described below in reference to FIG. 8C, a junction alternatively may be formed as an integral portion of a pre-bent anchor midportion. Referring to FIG. 2N, a close-up view of a saddle-shaped needle receiving structure (10) formed into the distal end of the pre-bent anchor midportion (4) shows a valley-like passageway (106), akin to the “spine” of a canoe, that is formed into the prebent anchor midportion (4) to assist in guiding and constraining the motion of the associated needle (40) tip (44). FIG. 2O illustrates an alternate view of a saddle-shaped needle receiving structure (10) formed into the distal end of the pre-bent anchor midportion (4). Referring to FIG. 2P, with the junction assembly (64) and flexible distal portion (2) hidden, a close-up orthogonal view highlights the saddle-shaped needle receiving structure (10) formed into the distal end of the pre-bent anchor midportion (4) and associated needle (40) which is movably coupled thereto. Also shown is the load assisting member (48) in a deployed out position. FIG. 2Q illustrates a prebent anchor midportion (4) without an associated flexible distal portion (2) or movably coupled needle (40). A port (104) and lumen (102) are shown formed into the midportion (4) using techniques such as laser cutting, to accommodate a needle (40) as described above. A load assisting member hinge coupling slot (108) may be similarly formed. The valley (106) and saddle-like (10) geometries may be created by mechanically manipulating an otherwise straight or bent piece of tubing to yield a fairly atraumatic outer geometry. Alternatively, such features may be laser cut into a piece of tubing, and any sharp edges may be polished away and/or rolled over to provide for atraumatic tissue interfacing. FIG. 2R shows an underside view of a similar structure with lasercut features for a load assisting member (48) to be moved into upon insertion or retraction of the pre-bent anchor midportion (4); another lasercut feature comprises a small cutout feature or aperture (88) created for a tensile element (50) to be passed through en route to coupling to a distal end of a load assisting member (48), as described below. FIG. 2S illustrates a close-up view of a suitable load assisting member (48) with a coupling hinge member (56) that may be coupled to the pre-bent anchor midportion (4) at the coupling slot (108). A tubular body (54) coupled to a distal plug member (52) may be rotatably connected to the hinge member (56) with a pivot joint pin (74). FIG. 2T illustrates a similar grouping of elements without the main tubular body (54) shown to illustrate coupling of a tension element (50) to a distal structure such as the distal plug member (52), with a configuration designed to cause rotation (90) of the load assisting member (48) about the pivot joint pin (74) axis under tension through the tension element (50). The load assisting member (48) and associated anchor midportion (4) may be intercoupled with a spring that is biased to maintain a closed configuration until a tensile load is applied in the tension member (50), after which deployment outward may occur and remain until the tensile load is released.

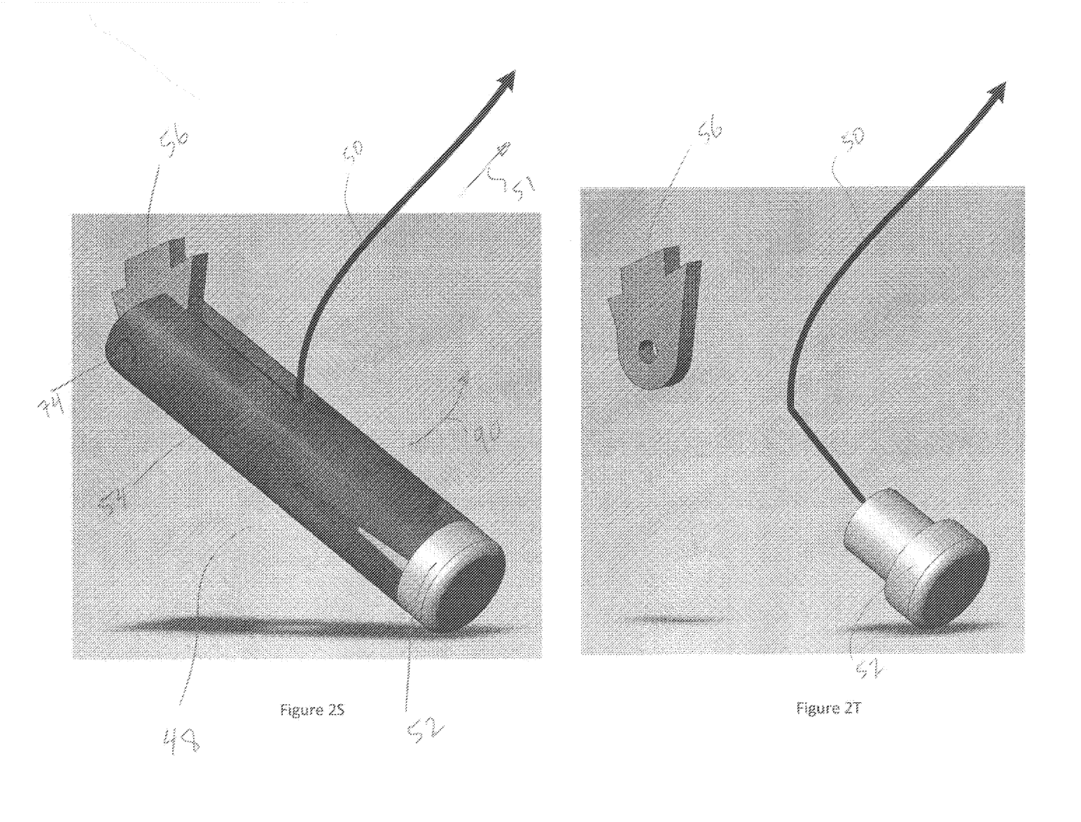

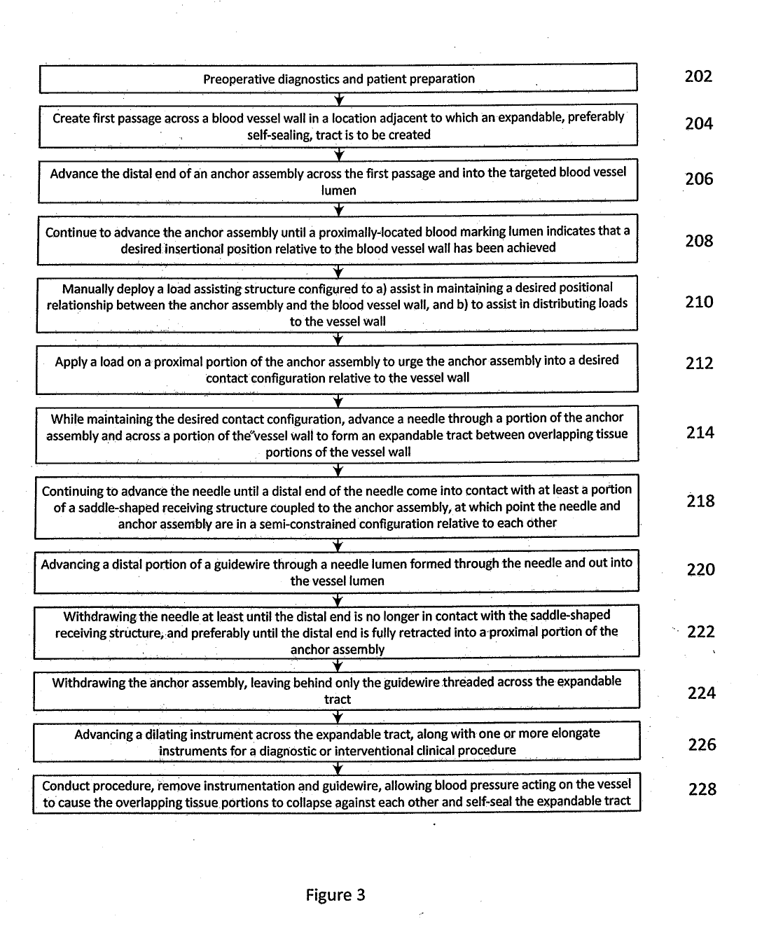

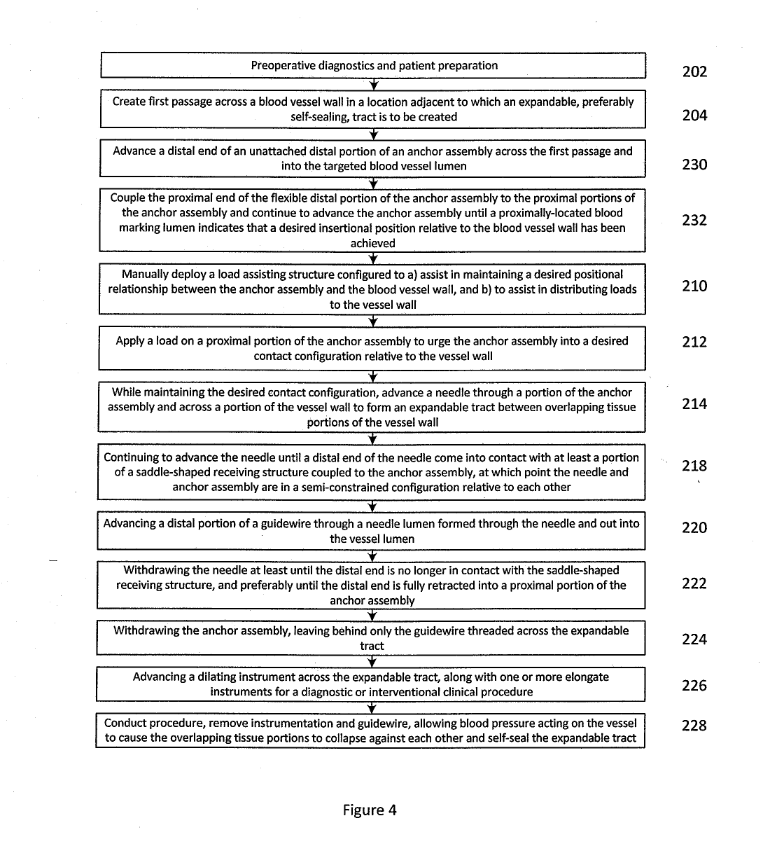

Referring to FIGS. 3 and 4, various embodiments of procedures utilizing the subject technology are illustrated. Referring to FIG. 3, after diagnostics and patient preparation for a diagnostic, interventional, and/or surgical procedure (202), a first passage may be created across a wall of a blood vessel and adjacent skin and subdermal tissue adjacent a location wherein an expandable, preferably self-sealing tract is to be created and utilized in a surgical procedure (204). With the first passage created, a distal end of an anchor assembly (the assembly generally comprising a flexible distal portion, a pre-bent midportion, and a proximal portion for manipulation and needle coupling) may be advanced across the first passage and into the targeted blood vessel (206). Advancement may be continued until a desired insertion configuration is achieved; this may be determined utilizing a blood marking configuration with a distally located port fluidly coupled to a proximal blood marking port (208). A load-assisting structure may be deployed (210) and utilized in loading the anchor assembly into a desired contact configuration with the associated vessel wall (212). While maintaining this desired contact configuration, a needle may be advanced at a carefully controlled trajectory across the vessel wall to create an expandable tract between two overlapping portions of the tissue wall (214). The needle advancement may be continued until a needle distal end contacts at least a portion of a constraining saddle-shaped receiving structure of the anchor assembly (218). While keeping the needle in place, a guidewire or other flexible elongate member may be inserted through a working lumen defined through the needle (220). The needle may then be withdrawn (222), followed by withdrawal of the anchor assembly (224), leaving behind only the guidewire placed across the expandable tract. This this self-sealing expandable tract created, a diagnostic and/or surgical procedure may be conducted using the tract, for example, by inserting a dilating instrument, such as a tapered introducer catheter, across the tract, along with other pertinent instruments (226) and conducting the diagnostic and/or interventional procedure. With the procedure completed, the instrumentation, guidewire, and dilating instrument may be removed in various orders (228), with removal of the last allowing blood pressure acting on the vessel to cause the overlapping tissue portions to collapse against each other and “self seal” the expandable tract. For example, in one embodiment, the surgical instrumentation may first be removed, followed by the dilator, leaving only the guidewire to be finally removed for self-sealing. In another embodiment the dilator may be last removed. In another embodiment, all may be removed together to effect self sealing of the tract.

The embodiment of FIG. 4 differs from that of FIG. 3 in that the flexible distal end of the anchor assembly is inserted into the lumen unattached from the other portions of the anchor assembly (230), after which it is coupled with the remainder of the anchor assembly (232) with the distal portion in situ.

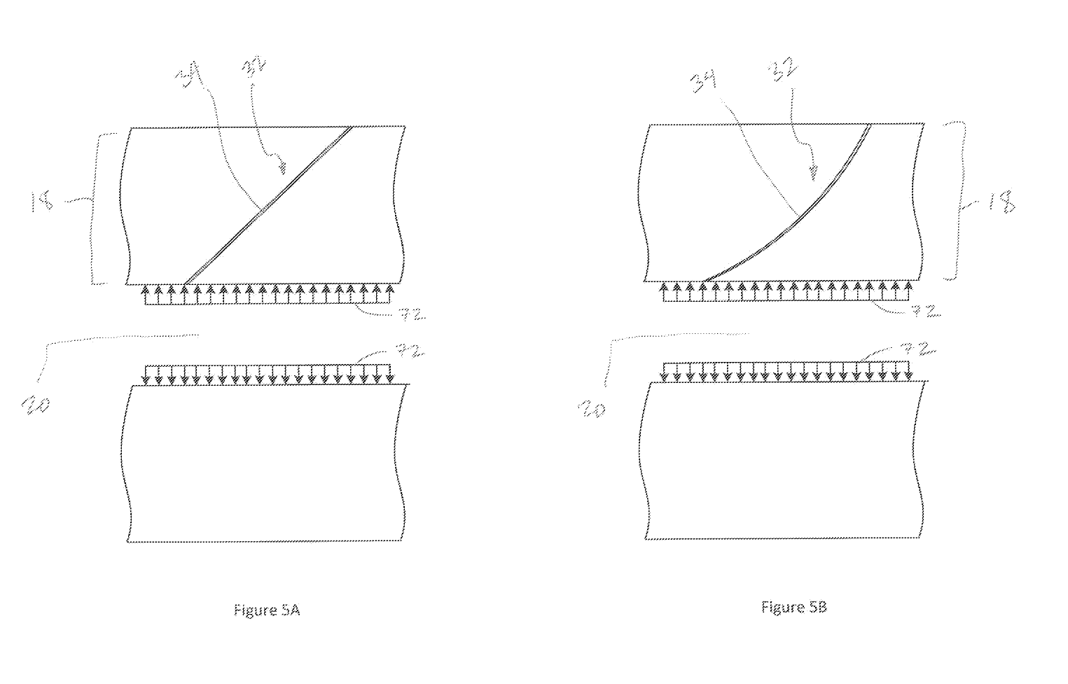

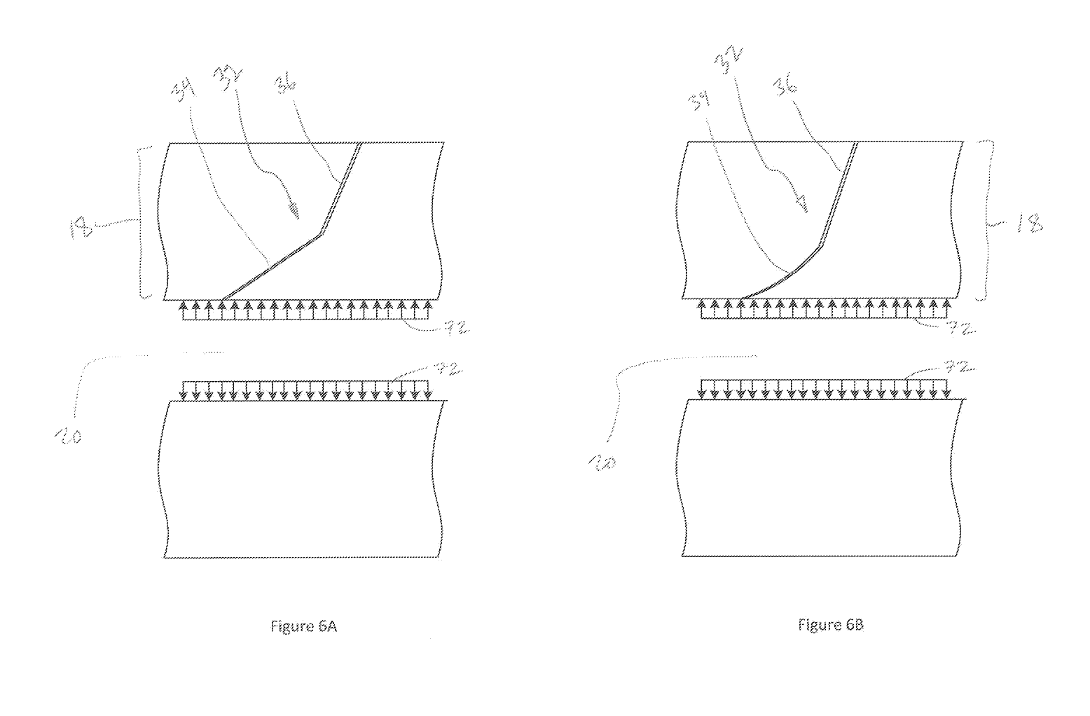

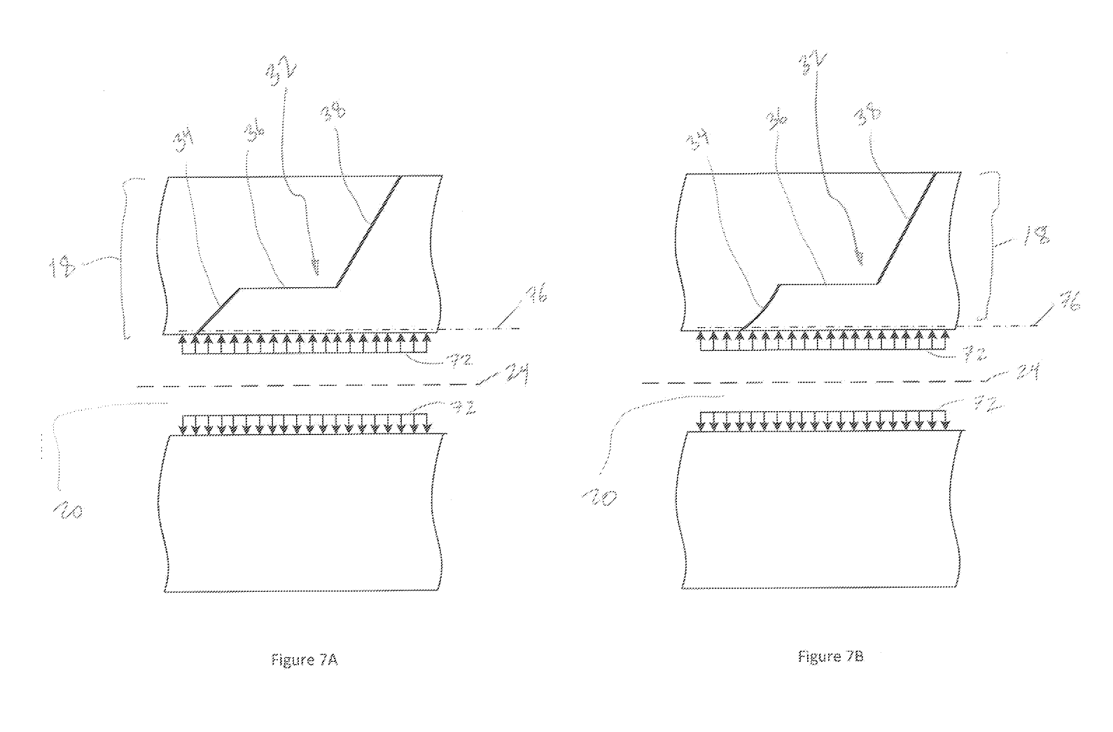

Referring to FIGS. 5A-7B, various illustrations of partial cross sections of vessel walls (18) are shown to depict various expandable tract (32) configurations that are within the scope of the present invention. Referring to FIG. 5A, a single segment (34) expandable tract has been created that is substantially straight relative to the lumen (20) of the vessel. Pressure (72) from pressurized blood within the lumen will act to self-seal the overlapping tissue portions bisected by the angled expandable tract (32). FIG. 5B features a single segment (34) self-sealing expandable tract (32) formed in an arcuate shape. Such an arcuate shape maybe created for an entire expandable tract or segment thereof using techniques such as a steerable needle, a needle with a predetermined insertion trajectory profile, and/or a needle with a cutting tip that results in an arcuate trajectory through tissue. Referring to FIGS. 6A, two segment (34, 36) expandable self-sealing tract configurations are depicted having straight and/or arcuate segments. Three segment (34, 36, 38) configurations of straight or nonstraight segments are shown in FIGS. 7A and 7B. To assist with the self-sealing action of the overlapping tissue portions bisected by the tract, the general trajectory of segments closer to the lumen (20) may be more closely parallel to the lumen longitudinal axis (shown as element 24 in FIG. 7B) or an axis parallel to the nearby vessel wall (shown as element 76 in FIG. 7B).

Referring to FIGS. 8A-8C, other embodiments of anchor assemblies are depicted with and without other associated elements, such as movably coupled needles. As shown in FIG. 8A, an anchor assembly is depicted comprising a saddle-shaped distal aspect (10) of a pre-bent midportion (4), wherein a relatively large elongate portion (112) of the midportion (4) is formed into a “valley” or saddle-shaped proximal continuation of the more distal saddle-shaped feature (10). This proximal extension of the constraining geometry may be utilized to further guide a needle (40) as such needle is inserted to create an expandable tract. In the depicted embodiment, the proximal extension (112) of the contraining valley-like geometry is configured to contact, and thereby geometrically constrain from lateral movement, between about ¼ and about ½ of the cross sectional geometry of portions of the needle (40) which may be interfaced therewith. As with the saddle-shaped structure depicted above, for example, in FIG. 2N, at least a portion of the saddle-shaped structure may be formed using laser cutting techniques to remove one or more portions of a tubelike structure, subsequent to which any edges left from cutting may be made relatively atraumatic by smoothing such edges or rolling the material local thereto. Referring to FIG. 8B, another embodiment of an anchor assembly is depicted wherein the distal saddle-shaped structure (10) of the pre-bent midportion (4) is formed by deformation of a tubular element, thereby producing a pre-bent midportion that is double-walled throughout its length (with the exception of cutouts for elements such as a needle to pass through), and that has a distal aspect (10) with an essentially uninterrupted and rounded surface, which may be desirable from an atraumatic insertion perspective. FIG. 8C depicts an embodiment similar to that depicted in FIG. 8B, with the exception that the junction member (64) shown in FIG. 8B, which may be coupled to the pre-bent midportion (4) during device assembly, is replaced in the embodiment of FIG. 8C with an all-in-one configuration, wherein the distal aspect of the pre-bent midportion (4) of the embodiment of FIG. 8C comprises a junction fitting and saddle-shaped, or valley-shaped, constraining structure (116) that are formed from the same piece of material (118), such as by controlled deformation and/or machining of a tubular structure. Such a configuration may be preferred for ease of manufacture and/or enhanced or more homogeneous device bending modulus reasons.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A method for forming an expandable tract across a wall of a blood vessel, comprising:
a. creating a first passage across the wall with a sharpened member at a first angle relative to a lumen longitudinal axis defined by a lumen of the blood vessel in the region adjacent the first passage;
b. advancing an anchor assembly through the first passage such that a distal portion of the anchor assembly is placed within the lumen of the blood vessel;
c. applying a force to the anchor assembly to position an adjacent portion of the blood vessel wall into a desired contact configuration relative to the anchor assembly;
d. while maintaining the desired contact configuration, advancing a needle through a portion of the anchor assembly and through a portion of the wall of the blood vessel to form an expandable tract between overlapping tissue portions of the vessel wall,
wherein a portion of the anchor assembly comprises a saddle-shaped needle receiving structure configured to receive and support the needle after it has been advanced across the portion of the wall to create the expandable tract.

2. The method of claim 1, wherein the sharpened member comprises a scalpel or a needle.

3. The method of claim 1, wherein the first passage is substantially straight in geometry.

4. The method of claim 1, wherein the anchor assembly comprises a flexible distal portion removably coupled to a substantially rigid proximal portion, and wherein the flexible distal portion is advanced through the first passage, after which it is coupled to the substantially rigid proximal portion and a force is applied to the anchor assembly to position the adjacent portion of the blood vessel wall into the desired contact configuration relative to the anchor assembly.

5. The method of claim 1, wherein the desired contact configuration is characterized in that at least a portion of the anchor assembly is oriented substantially parallel to the lumen longitudinal axis.

6. The method of claim 1, wherein the desired contact configuration is characterized in that at least a portion of the anchor assembly is oriented substantially parallel to an axis parallel to a portion of the vessel wall immediately adjacent the expandable tract.

7. The method of claim 1, further comprising controllably extending a load assisting structure from the anchor assembly before applying the force to the anchor assembly.

8. The method of claim 7, wherein controllably extending comprises rotating the load assisting structure about a pivot point relative to the anchor assembly.

9. The method of claim 7, wherein controllably extending comprises extending the load assisting member outward from an outer surface of the anchor assembly along a substantially straight axial pathway relative to the anchor assembly.

10. The method of claim 7, wherein controllably extending comprises extending the load assisting member outward from an outer surface of the anchor assembly along an arcuate pathway relative to the anchor assembly.

11. The method of claim 7, wherein controllably extending comprises manually applying a load to a proximal portion of the anchor assembly, at least a portion of such load being transferred to one or more members coupled to the load assisting structure.

12. The method of claim 1, wherein the expandable tract is substantially straight in geometry.

13. The method of claim 1, wherein the expandable tract comprises two longitudinal portions that are angled relative to each other.

14. The method of claim 13, wherein a distal portion leading to the lumen of the blood vessel is angled more steeply relative to the lumen longitudinal axis than is a more proximal portion.

15. The method of claim 1, further comprising advancing the needle until a distal portion of the needle comes into contact with at least a portion of the saddle-shaped needle receiving structure.

16. The method of claim 1, further comprising advancing a guidewire through the expandable tract.

17. The method of claim 16, wherein the needle is hollow, and wherein the guidewire is advanced through a needle lumen defined through the needle.

18. The method of claim 16, further comprising withdrawing the anchor assembly, leaving behind only the guidewire through the expandable tract.

19. The method of claim 18, further comprising advancing a dilating instrument across the expandable tract.

20. The method of claim 19, further comprising advancing one or more elongate instruments through the dilating instrument to conduct a portion of a diagnostic or interventional procedure.

21. The method of claim 20, further comprising withdrawing the one or more elongate instruments and dilating instrument and leaving a guidewire in place across the expandable tract.

22. The method of claim 21, further comprising withdrawing the guidewire to allow blood pressure acting on the vessel wall to cause the overlapping tissue portions to collapse against each other and self-seal the expandable tract.

23. The method of claim 20, further comprising withdrawing the one or more elongate instruments and the guidewire and leaving the dilating instrument in place across the expandable tract.

24. The method of claim 23, further comprising withdrawing the dilating instrument to allow blood pressure acting on the vessel wall to cause the overlapping tissue portions to collapse against each other and self-seal the expandable tract.

25. The method of claim 20, further comprising withdrawing the one or more elongate instruments, the dilating instrument, and the guidewire to allow blood pressure acting on the vessel wall to cause the overlapping tissue portions to collapse against each other and self-seal the expandable tract.
